SEQUENCE LISTING (2 sequences)

SEQ ID NO: 1 - protein, 901 aa, Homo sapiens
MEANQCPLVVEPSYPDLVINVGEVTLGEENRKKLQKIQRDQEKERVMRAACALLNSGGGVIRMAKKVEHPVEMGLDLEQSLRELIQSSDLQAFFETKQQGRCFYIFVKSWSSGPFPEDRSVKPRLCSLSSSLYRRSETSVRSMDSREAFCFLKTKRKPKILEEGPFHKIHKGVYQELPNSDPADPNSDPADLIFQKDYLEYGEILPFPESQLVEFKQFSTKHFQEYVKRTIPEYVPAFANTGGGYLFIGVDDKSREVLGCAKENVDPDSLRRKIEQAIYKLPCVHFCQPQRPITFTLKIVNVLKRGELYGYACMIRVNPFCCAVFSEAPNSWIVEDKYVCSLTTEKWVGMMTDTDPDLLQLSEDFECQLSLSSGPPLSRPVYSKKGLEHKKELQQLLFSVPPGYLRYTPESLWRDLISEHRGLEELINKQMQPFFRGILIFSRSWAVDLNLQEKPGVICDALLIAQNSTPILYTILREQDAEGQDYCTRTAFTLKQKLVNMGGYTGKVCVRAKVLCLSPESSAEALEAAVSPMDYPASYSLAGTQHMEALLQSLVIVLLGFRSLLSDQLGCEVLNLLTAQQYEIFSRSLRKNRELFVHGLPGSGKTIMAMKIMEKIRNVFHCEAHRILYVCENQPLRNFISDRNICRAETRKTFLRENFEHIQHIVIDEAQNFRTEDGDWYGKAKSITRRAKGGPGILWIFLDYFQTSHLDCSGLPPLSDQYPREELTRIVRNADPIAKYLQKEMQVIRSNPSFNIPTGCLEVFPEAEWSQGVQGTLRIKKYLTVEQIMTCVADTCRRFFDRGYSPKDVAVLVSTAKEVEHYKYELLKAMRKKRVVQLSDACDMLGDHIVLDSVRRFSGLERSIVFGIHPRTADPAILPNVLICLASRAKQHLYIFPWGGH

SEQ ID NO: 2 - DNA, 4862 bp, Homo sapiens
gtgcagtggcacgatcttggttcaccacaatctcgtctcgaaggctcaggtgattctctcacctcagccgcctgagtagctgggaccacagtttcagctgtgagttcaacatggaggcaaatcagtgccccctggttgtggaaccatcttacccagacctggtcatcaatgtaggagaagtgactcttggagaagaaaacagaaaaaagctgcagaaaattcagagagaccaagagaaggagagagttatgcgggctgcatgtgctttattaaactcaggaggaggagtgattcgaatggccaagaaggttgagcatcccgtggagatgggactggatttagaacagtctttgagagagcttattcagtcttcagatctgcaggctttctttgagaccaagcaacaaggaaggtgtttttacatttttgttaaatcttggagcagtggccctttccctgaagatcgctctttcaagccccgcctttgcagcctcagttcttcattatacctgtagatctgagacctctgtgcgttccatggactcaagagaggcattctgtttcctgaagaccaaaaggaagccaaaaatcttggaagaaggaccttttcacaaaattcacaagggtgtataccaagagctccctaactcggatcctgctgacccaaactcggatcctgctgacctaattttccaaaaagactatcttgaatatggtgaaatcctgccttttcctgagtctcagttagtagagtttaaacagttctctacaaaacacttccaagaatatgtaaaaaggacaattccagaatacgtccctgcatttgcaaacactggaggaggctatctttttattggagtggatgataagagtagggaagtcctgggatgtgcaaaagaaaatgttgaccctgactctttgagaaggaaaatagaacaagccatatacaaactaccttgtgttcatttttgccaaccccaacgcccgataaccttcacactcaaaattgtggatgtgttaaaaaggggagagctctatggctatgcttgcatgatcagagtaaatcccttctgctgtgcagtgttctcagaagctcccaattcatggatagtggaggacaagtacgtctgcagcctgacaaccgagaaatgggtaggcatgatgacagacacagatccagatcttctacagttgtctgaagattttgaatgtcagctgagtctatctagtgggcctccccttagcagaccagtgtactccaagaaaggcctggaacataaaaaggaactccagcaacttttattttcagtcccaccaggatatttgcgatatactccagagtcactctggagggacctgatctcagagcacagaggactagaggagttaataaataagcaaatgcaacctttctttcggggaattttgatcttctctagaagttgggctgtggacctgaacttgcaggagaagccaggagtcatctgtgatgctctgctgatagcacagaacagcacccccattctctacaccattctcagggagcaggatgcagagggccaggactactgcactcgcactgcctttactttgaagcagaagctagtgaacatggggggctacaccgggaaggtgtgtgtcagggccaaggtcctctgcctgagtcctgagagcagcgcagaggccttggaggctgcagtgtctccgatggattaccctgcgtcctatagccttgcaggcacccagcacatggaagccctgctgcagtccctcgtgattgtcttactcggcttcaggtctctcttgagtgaccagctcggctgtgaggttttaaatctgctcacagcccagcagtatgagatattctccagaagcctccgcaagaacagagagttgtttgtccacggcttacctggctcagggaagaccatcatggccatgaagatcatggagaagatcaggaatgtgtttcactgtgaggcacacagaattctctacgtttgtgaaaaccagcctctgaggaactttatcagtgatagaaatatctgccgagcagagacccggaaaactttcctaagagaaaactttgaacacattcaacacatcgtcattgacgaagctcagaatttccgtactgaagatggggactggtatgggaaggcaaaaagcatcactcggagagcaaagggtggcccaggaattctctggatctttctggattactttcagaccagccacttggattgcagtggcctccctcctctctcagaccaatatccaagagaagagctcaccagaatagttcgcaatgcagatccaatagccaagtacttacaaaaagaaatgcaagtaattagaagtaatccttcatttaacatccccactgggtgcctcgaggtatttcctgaagccgaatggtcccagggtgttcagggaaccttacgaattaagaaatacttgactgtggagcaaataatgacctgtgtggcagacacgtgcaggcgcttctttgataggggctattctccaaaggatgttgctgtgcttgtcagcaccgcaaaagaagtggagcactataagtatgagctcttgaaagcaatgaggaagaaaagggtggtgcagctcagtgatgcatgtgatatgttgggtgatcacattgtgttggacagtgttcggcgattctcaggcctggaaaggagcatagtgtttgggatccatccaaggacagctgacccagctatcttacccaatgttctgatctgtctggcttccagggcaaaacaacacctgtatatttttccgtggggtggccattaggaagaactccaaatcaaaatgctatgtaaatgtctatgggtgacagtctgctgatggtagaaacctttctttttagttcacaagtcagagatttggacggagctgacacaaagagtttggagctcccccatttctggctctcctttcaggggttccttccccaacccttttcagcagcggtggctgccccccattctgacccctgactcttccagccagaaagatggtggttttctaaaggaactttagctgtcctgcacaatgccgatctgtgtcttgcattttgggtaaaagccataaaaataagaaactcagcctgtggcctttctttcttccaaggctgggcttctttttttaagtgacttcatgcagtttgttgcttttaaaaatttgtccagaatcgttttctgcagaagcatggtctgttaggagcttactggccgtagcagaagcaattgtttcctgaattcttgacatttatctttgctgtattcatttagggcttgggagagtccgaagataattcagtcactgtcagattaataattctgtcaggacaaagaataccgttatgattatttaatcctttaaaattgtggtctccagagcttgttctcagaatggcccagaccaagccttaattgtgatagtgaatattaatggtcactttaaggagaaattataggccaagatgaaatgaacataaacctgtttgccctggctttcagtggaagatgatattagagaccaaaatctggttctgaaggtgtgtatcagccctaaggtgaaccagacttgggaaagattgtctttaaaaatcaatgagtttatgttttaacttctcagcttagttctatgcattgctctataacacacctagttaagttttatgttattcttgaactgtgattttttttctatttactttcatggtttggtgggccattgttatggactgaatgtttgtgtcccacccttcacccccaaattcccgtgttgaagccccaacctgcactgtggagctggggctgctaaggaagtaattaaggttacatgaagtcatggtggggctctgatctgctaaggttggtgtccttatagggagagaccccagagagcttgttccctccctccctgtgcatgcaaacaagagggcatgggagcacacagagagatggcagccacctacaagccaagaggagaagcctcacaatcaaactctcgctgctggcgagagtcttggactctgtcttggacttccagcctccagactgtgagaaacaaatttctgttgtttcagcttctcagtctctggtgttttgttattgcagcctgagaacacagctgtacgattatttgtcaaacagaaaacactgatacttaacaatgctaatgcaattatttatttgcttttcagtctctacaaaacgttctaaaacactaatctaaatattaacagtaaaatatttgcataactaatggaaactaagaaatcatatgaccaatatttcacttattggtaatcttactctactgatttccccccagactgtgatttttgaacttccttgcctttctcctgtctttctgtgtttattcatggaattccagttatctgggcttgaaattgcaggctctcctaacttaagcaaaatctgacagatcagcaaaatgagataaatgtttcttttttctttctgactgcattaaatcagatacaactcagcattaaaaagctatctttgtaaatgttgttactaataaattagtcttataagatccctggactttggagttgttgcaatgtctttgagagtaattctttaaaagtctaatttcgactggttgtatctctttatgatttattgccccactaacaacatttgaaacaatataatattttaaaatgtataaataattatgaatttttgtttagaacaaagaggattactgatatttgtttccctatgaatggcaaaaggtttagcttactactgcatttctgttttaaataaaaagttgagagtttgtgtctcattaaactg

(19) United States
(12) Patent Application Publication
(10) Pub. No.: US 2017/0314077 A1
(43) Pub. Date: Nov. 2, 2017

(54) SLFN11 AS BIOMARKER FOR AML

(71) Applicant: BERGENBIO AS, Bergen (NO)

(72) Inventors: David Robert MICKLEM, Bergen (NO); Monica HELLESOY, Bergen (NO); Linn Hodneland NILSSON, Bergen (NO)

(73) Assignee: BERGENBIO AS, Bergen (NO)

(21) Appl. No.: 15/525,623

(22) PCT Filed: Nov. 13, 2015
(86) PCT No.: PCT/EP2015/076603

§ 371 Date: May 10, 2017

(30) Foreign Application Priority Data
Nov. 14, 2014 (GB) 1420239.4

(51) Int. Cl. C12Q 1/68; G01N 33/50; A61K 31/502; G01N 33/574; C07K 14/47

(52) U.S. Cl. CPC C12Q 1/6886; C07K 14/4703; G01N 33/5088; G01N 33/574; G01N 33/57496; G01N 33/5011; A61K 31/502; C12Q 2600/106; C12Q 2600/118; C12Q 2600/136; G01N 2333/4704; C12Q 2600/158

(57) ABSTRACT

The use of Slfn11 as a biomarker for detecting the occurrence of epithlial-to-mesenchymal transition (EMT) in a subject, and the use of Slfn11 modulators to treat cancer is disclosed herein. Also disclosed are various methods for detecting the occurrence of epithelial-to-mesenchymal transition (EMT) in a subject by measuring Slfn11 expression and/or activity.

### FIELD OF INVENTION

This invention relates to the fields of drug development and cancer treatment. In particular this invention relates to the field of protein kinases and more particularly to methods of prognosing and treating cancer.

### BACKGROUND TO THE INVENTION

### Slfn11

Slfn11 (Schlafen family member 11) is a member of the Schlafen family of proteins, characterised by the presence of a Slfn-box domain of unknown function. The Slfn box lies near an AAA domain, which has been demonstrated in other proteins to be an ATP/GTP-binding motif. Slfn11 is a member of the group III Slfn's which have an additional Slfn-specific “SWADL”-domain of unknown function, a nuclear localisation signal and an RNA-helicase-like motif. This constellation of domains has led to suggestions that group III SLFN proteins are involved in processing of RNA in the nucleus [Mavrommatis et al., J Interferon Cytokine Res. April 2013; 33(4): 206-210].

SLFN11 is an interferon (IFN)-induced antiviral protein which acts as an inhibitor of retrovirus protein synthesis. The protein specifically abrogates the production of retroviruses such as human immunodeficiency virus 1 (HIV-1) by acting as a specific inhibitor of the synthesis of retroviruses encoded proteins in a codon-usage-dependent manner [Li M. et al., Nature. 2012; 491(7422):125-128]. The mechanism by which this inhibition occurs was described, and appears to involve blocking expression of viral proteins in a codon usage-defined manner (Li and others 2012). Thus, human SLFN11 appears to play a key role in the control of HIV infection in humans, and it is possible that its selective targeting may lead to the development of new antiviral drugs.

Slfn11 has also been found to bind to tRNAs and exploits the unique viral codon bias towards A/T nucleotides. The exact inhibition mechanism is unclear: Slfn11 may either sequester tRNAs, prevent their maturation via post-transcriptional processing or accelerate their deacylation. Slfn11 does not inhibit reverse transcription, integration or production and nuclear export of viral RNA.

Other recent studies have demonstrated that SLFN11 has an important role in sensitizing malignant cells to topoisomerase inhibitors [Barretina J. et al. Nature. 2012; 483(7391):603-607], [Zoppoli G. et al., Proc Natl Acad Sci USA. 2012; 109(37):15030-15035] as well as alkylating agents and other DNA-damaging agents [Zoppoli G. et al., Proc Natl Acad Sci USA. 2012; 109(37):15030-15035].

A role for Slfn11 in cell cycle arrest and/or induction of apoptosis in response to exogenously induced DNA damage has also been suggested.

### Acute Myeloid Leukemia (AML)

Acute myeloid leukemia (AML) is a clonal disease of hematopoietic progenitors that is characterized by numerous heterogeneous genetic changes that alter the cells' normal mechanisms of proliferation, differentiation and cell death (Burnett et al., 2011). Currently, most AML patients are treated with chemotherapy such as Cytarabine. Apart from the BCR-Abl targeting drug Gleevec for treatment of chronic myelogenous leukemia (CML) and the differentiation-inducing drug ATRA for treatment of acute promyelocytic leukemia (APL), targeted therapy has so far been extensively lacking in the field of leukemia. This despite AML being a well characterized disease with several mutated oncogenes that could potentially be therapeutic targets (Haferlach, 2008).

### Axl

Axl is a member of the TAM (Tyro-Axl-Mer) family of transmembrane receptor tyrosine kinases (RTK), which regulates a large range of cellular responses (Hafizi and Dahlback, 2006). Axl, in particular, is expressed in embryonic tissues and plays a role in neural and mesenchymal development. Increased Axl expression or activation is a characteristic of various disease states.

Studies have shown that Axl plays a number of different roles in tumour formation. Axl is a key regulator of angiogenic behaviours including endothelial cell migration, proliferation and tube formation. Axl is also required for human breast carcinoma cells to form a tumour in vivo, indicating that Axl regulates processes that are vital for both neovascularisation and tumorigenesis (Holland S. et al, Cancer Res 2005; 65 (20), Oct. 15, 2005).

The activity of Axl receptor tyrosine kinase is positively correlated with tumour metastasis. More specifically, studies have shown that Axl enhances expression of MMP-9, which is required for Axl-mediated invasion. Axl promotes cell invasion by inducing MMP-9 activity through activation of NF-BK and Brg-1 (Tai, K-Y et a/, Oncogene (2008), 27, 4044-4055).

Axl is overexpressed in human glioma cells and can be used to predict poor prognosis in patients with Glioblastoma Multiforme (GBM) (Vajkoczy P. et a/, PNAS, Apr. 11, 2006, val 103, no. 15, 5799-5804; Hutterer M. eta/, Clinical Cancer Res 2008; 14 (1) Jan. 1, 2008;). Axl is also relatively overexpressed in highly invasive lung cancer cell lines compared to their minimally invasive counterparts (Shieh, Y-S eta/, Neoplasia, val 7, no. 12, December 2005, 1058-1064). Axl is therefore believed to play an important role in tumour invasion and progression.

Likewise, Axl is expressed in highly invasive breast cancer cells, but not in breast cancer cells of low invasivity. More specifically, inhibition of Axl signalling (by dominant-negative Axl mutant, an antibody against the extracellular domain of Axl, or by short hairpin RNA knockdown of Axl) decreased the motility and invasivity of highly invasive breast cancer cells. Small molecule Axl inhibitors interfered with motility and invasivity of breast cancer cells. Thus, Axl is understood to be a critical element in the signalling network that governs the motility/invasivity of breast cancer cells (Zhang, Y-X et al., Cancer Res 2008; 68 (6), Mar. 15, 2008).

The Axl pathway has also been implicated in organ degenerative diseases such as fibrosis of the liver and/or kidney. For example, it has been shown in mice that a deficiency of the Axl ligand Gash prevents liver inflammation and fibrosis [Fourcot, A. et al., Am J Physiol Gastrointest Liver Physiol 300: G1043-G1053, 2011].

Axl is also upregulated and constitutively active in human AML (Park et al., 2013). Furthermore, Axl was recently identified as an independent prognostic marker and a potential new therapeutic target in AML (Ben-Batalla et al., 2013). Axl upregulation is also reported to result in constitutive activation of the receptor tyrosine kinase Flt3, both in the wild type form and in Flt3 with an internal tandem duplication (ITD) mutation (Park et al., 2013). About 30% of AML patients carry an ITD mutation in the Flt3 kinase. Flt3-ITD is an independent prognostic marker and is coupled to poor prognosis and therapeutic resistance in AML (Vardiman et al., 2009). So far, no Flt3 inhibitor has been successfully implemented in the clinical treatment of AML (Levis, 2013).

### The Epithelial-Mesenchymal Transition (EMT)

Epithelial tissues make up one of the four basic tissue types of the body, along with connective tissue, muscle and nervous tissue. Epithelial cells are characterised by a tendency to form into sheets of polarised cells held together by strong intercellular junctions. As a consequence of this, epithelial cells are not able to move freely and show little migration compared to other cell types. In contrast, mesenchymal-like cells (e.g. fibroblasts) lack strong intercellular junctions and can move as individual cells. They can be highly motile and able to migrate through the extracellular matrix.

The Epithelial-Mesenchymal Transition (EMT) is a natural cellular program in which individual epithelial cells lose the gene expression patterns and behaviours characteristic of epithelial cells and instead begin to look, behave and express genes typical of mesenchymal cells. In so doing they lose adhesion and apical-basal polarity and gain the ability to migrate and invade the extracellular matrix. EMT is not irreversible. A mirror process called Mesenchymal-Epithelial Transition (MET) results in the loss of mesenchymal characteristics and re-establishment of cell-cell adhesion and apical-basal polarity.

EMT is especially important during embryonic development. It plays a fundamental role in gastrulation, where an embryo consisting of a single epithelial cell layer develops into one with the three classical germ layers, ectoderm, mesoderm and endoderm. Slightly later in vertebrate development, EMT gives rise to the neural crest cells. These cells migrate throughout the embryo and give rise to many different structures including ganglia of the peripheral nervous system, bone and cartilage of the face and head, pigment cells and glial cells. Further rounds of MET and EMT are essential for the formation of internal organs from both the mesoderm and endoderm.

### EMT and Disease

In contrast to its importance during embryonic development, the EMT program is seldom activated in healthy adults. It is, however, induced in response to inflammation following injury or disease: EMT plays a role in wound healing and tissue repair, and occurs during organ degenerative disease (e.g. fibrotic diseases such as renal, pulmonary or hepatic fibrosis).

EMT is also increasingly understood to play a key role in cancer metastasis. Carcinomas are epithelial cancers, and, in order for metastasis to occur, individual cells must escape the primary tumour and undergo a series of migrations. These include migration from the primary tumour into the local circulatory or lymphatic system, and extravasation from the vasculature and establishment at the site of metastasis. There is now good and growing evidence that interactions between tumour cells and their microenvironment can lead to induction of EMT in some of the tumour cells. The resulting increased cell migration and invasion potential of these cells then enhances the likelihood of a metastasis becoming established. The receptor tyrosin kinase Axl, which is a chronic myelogenous leukemia-associated oncogene, has recently been shown to be an essential EMT-induced effector in the invasion-metastasis cascade (WO2010/103388).

As well as this role in increasing metastatic potential, the EMT program has recently been linked with Cancer Stem Cells (CSCs). These cells were first identified in acute myelocytic leukemia (AML), and have been postulated to represent a subset of tumour cells with stem cell characteristics—i.e. the ability to give rise to all the cell types found in a particular cancer, and thus the ability to form a new tumour. Although they may represent only a tiny fraction of the cells in a tumour, CSCs are thought to be particularly resistant to existing anti-cancer drugs. Even though drug treatment may kill the vast majority of cells in the tumour, a single surviving CSC can therefore lead to a relapse of the disease. Recent evidence suggests an overlap between EMT and CSC phenotypes, suggesting that EMT may also play a role in recurrence of cancer after chemotherapy and the development of drug-resistant tumours in cancer, including AML.

Robust biomarkers for the EMT phenotype would be useful in identifying patients at particular risk of developing metastatic or drug-resistant cancer, while novel drugs that target cells that have undergone EMT will reduce metastasis and relapse following conventional therapy.

### SUMMARY OF THE INVENTION

The present inventors have found that expression of Slfn11 is regulated by Axl receptor tyrosine kinase signalling. More specifically, it has been found that levels of Slfn11 expression are reduced in a dose-dependent manner in an acute myeloid leukemia (AML) cell line upon exposure to the Axl inhibitor BGB324/R428. Accordingly, expression of Slfn11 can be used as an indicator of Axl receptor tyrosine kinase signalling in the applications described herein.

The demonstration of the link between Axl receptor tyrosine kinase signalling and Slfn11 expression identifies Slfn11 as part of the Axl signalling cascade. Thus, the current studies have identified Slfn11 as a potential new target for intervention and treatment of conditions in which the Axl pathway plays a role.

According to one aspect of the invention, there is provided a method of selecting a pharmaceutical compound useful for the prevention, inhibition or treatment of an Axl-related condition the method comprising providing a group of candidate pharmaceutical compounds for testing, testing the effect of candidate pharmaceutical compounds in a test system, and selecting a candidate pharmaceutical compound on the basis of inhibiting Slfn11 expression. Axl-related conditions include, but are not limited to, solid cancer tumors, including, but not limited to, breast, renal, endometrial, ovarian, thyroid, and non-small cell lung carcinoma, melanoma, prostate carcinoma, sarcoma, gastric cancer and uveal melanoma; liquid tumors, including but not limited to, leukemias (particularly myeloid leukemias) and lymphomas; endometriosis, vascular disease/injury (including but not limited to restenosis, atherosclerosis and thrombosis), psoriasis; visual impairment due to macular degeneration; diabetic retinopathy and retinopathy of prematurity; kidney disease (including but not limited to glomerulonephritis, diabetic nephropathy and renal transplant rejection), rheumatoid arthritis; osteoarthritis, osteoporosis and cataracts; fibrosis (including but not limited to lung fibrosis and liver fibrosis).

Alternatively the invention provides a method of selecting a candidate pharmaceutical compound useful in the treatment of metastatic or drug resistant cancer, the method comprising providing a group of candidate pharmaceutical compounds for testing, testing the effect of candidate pharmaceutical compounds in a test system, and selecting a candidate pharmaceutical compound on the basis of its inhibition of Slfn11 expression.

According to another aspect there is provided a method of selecting a candidate pharmaceutical compound useful in the prevention or inhibition of EMT, the method comprising providing a group of candidate pharmaceutical compounds for testing, testing the effect of candidate pharmaceutical compounds in a test system, and selecting a candidate pharmaceutical compound on the basis of inhibiting Slfn11 expression.

It is highly advantageous to be able to determine effective levels of a candidate pharmaceutical compound in an in vitro test system in order to predict in vivo responses. This facilitates determination of effective minimum dosage levels of a pharmaceutical compound and also the validation of drug targets in a dose-dependent manner. A particularly useful approach to predicting in vivo responses to a pharmaceutical is through conditional selective knockout of a target gene through RNA interference. The effective generation of nucleotides for use in such methods is described in WO2009/082488.

According to another aspect of the invention there is provided a method of selecting a candidate pharmaceutical compound useful in the prevention, inhibition or treatment of an Axl-related condition, the method comprising selectively reducing expression of Axl in a test cell, contacting the test cell with the candidate pharmaceutical compound and determining the effect of the candidate pharmaceutical compound on inhibition of Slfn11 expression.

According to a further aspect of the invention there is provided a method of selecting a compound useful in the prevention, inhibition or treatment of an Axl-related condition, the method comprising selectively reducing expression of Axl in an in vitro test system to a low level contacting the test system with a candidate pharmaceutical compound, and selecting candidate pharmaceutical compounds which inhibit Slfn11 expression.

According to a further aspect of the invention there is provided a method of identifying a subject having an Axl-related condition, the method comprising assessing the level of expression or activity of Slfn11 in the subject, or in a sample derived from the subject. Generally, the level of expression or activity in a subject or in a sample derived from a subject may be determined relative to a control sample, as described herein.

According to a further aspect of the invention there is provided a method of identifying a subject having a particular risk of developing metastatic or drug-resistant cancer, the method comprising assessing the level of expression or activity of Slfn11 in the subject, or in a sample derived from the subject, an increased level of Slfn11 expression or activity indicating an increased risk of the subject of developing metastatic or drug-resistant cancer.

According to a further aspect of the invention there is provided a method of identifying the presence of a Cancer Stem Cell in a subject, the method comprising determining the level of Slfn11 expression or activity in the subject, or in a sample derived from the subject, increased expression or activity of Slfn11 indicating the existence of a Cancer Stem Cell (CSC).

According to a further aspect of the invention there is provided a method of identifying a subject undergoing EMT, the method comprising determining the level of Slfn11 expression or activity in the subject, or in a sample derived from the subject, an increase in expression or activity of Slfn11 indicating the occurrence of EMT.

According to a further aspect of the invention there is provided a method of prognosing a cancer-related outcome in a subject, the method comprising assessing Slfn11 activity or expression in the subject, or in a sample derived from the subject.

In some embodiments, an increase in Slfn11 activity or expression relative to a control sample is indicative of increased susceptibility to treatment with a cancer therapeutic agent, for example an agent capable of inhibiting or reversing EMT, or another chemotherapeutic agent as defined herein. Agents capable of inhibiting or reversing EMT are described herein e.g. an Akt3 inhibitor or an Axl inhibitor.

According to a further aspect of the invention there is provided a method of identifying Axl activity, the method comprising determining the level of Slfn11 expression or activity in the subject, or in a sample derived from the subject, increased activity or expression of Slfn11 correlating with Axl activity.

In some embodiments, the level of expression of Slfn11 is assessed by determining the copy number of the gene encoding Slfn11 relative to a control sample, wherein an increase in the copy number indicates an increased level of expression of Slfn11. Copy number (i.e. gene duplication events) may be determined using standard techniques known in the art, e.g. using a DNA chip as described in Jiang et al. (Jiang Q, Ho Y Y, Hao L, Nichols Berrios C, Chakravarti A. Copy number variants in candidate genes are genetic modifiers of Hirschsprung disease. PLoS One. 2011; 6(6)).

In some embodiments, the level of expression of Slfn11 is assessed by determining the level of Slfn11 protein or mRNA. Methods for determining protein and mRNA expression levels are well known in the art, and described herein.

According to another aspect of the invention there is provided a method of treating a subject having an Axl-related condition, the method comprising contacting the subject with an Slfn11 modulator, such as an activator or an inhibitor, or with a pharmaceutical compound selected as, or derived from, a candidate compound obtained by a method according to the first aspect of the invention.

Further aspects of the invention include a method of inhibiting EMT as subject, the method comprising contacting the subject with a compound capable of modulating, such as increasing or inhibiting, Slfn11 expression or activity.

A further aspect of the invention provides a method of inhibiting Cancer Stem Cells in a subject, the method comprising of contacting the subject with a compound capable of modulating Slfn11 expression or activity.

The invention also provides a method of preventing or inhibiting drug resistance in a subject having cancer, the method comprising contacting the subject with a compound capable of modulating Slfn11 expression or activity.

The invention also provides the use of a Slfn11 modulator in the treatment of an Axl related condition, such as cancer.

The invention also provides the use of a Slfn11 modulator in the inhibition of EMT.

The invention also provides a Slfn11 modulator for use in a method of treatment as described herein.

According to a further aspect of the invention there is provided the use of a compound capable of modulating Slfn11 activity or expression in the prevention, inhibition, or treatment of drug resistance in a subject having cancer, the method comprising contacting the subject with a compound capable of modulating Slfn11 activity or expression.

Slfn11 modulators identified by methods in accordance with the invention or used in methods or uses in accordance with the invention may be used as a monotherapy or in combination therapy with other cancer treatments as mentioned below.

Suitable chemotherapeutic agents include:

alkylating agents, including alkyl sulfonates such as busulfan;
nitrogen mustards such as chlorambucil, cyclophosphamide, estramustine, ifosfamide, mechlorethamine, melphalan, and uramustine, ethyleneimine derivatives such as thiotepa;
nitrosoureas such as carmustine, lomustine, and streptozocin, triazenes such as dacarbazine, procarbazine, and temozolamide, and
platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II); antimetabolites, including antifolates such as methotrexate, permetrexed, raltitrexed, and trimetrexate,
pyrimidine analogs such as azacitidine, capecitabine, cytarabine, edatrexate, floxuridine, fluorouracil, gemcitabine, and troxacitabine, and
purine analogs such as cladribine, chlorodeoxyadenosine, clofarabine, fludarabine, mercaptopurine, pentostatin, and thioguanine;
natural products, including antitumor antibiotics such as bleomycin, dactinomycin, mithramycin, mitomycin, mitoxantrone, porfiromycin, and anthracyclines such as daunorubicin, doxorubicin, epirubicin, idarubicin, and valmbicin,
mitotic inhibitors such as the vinca alkaloids vinblastine, vinvesir, vincristine, vindesine, and vinorelbine,
enzymes such as L-asparaginase and PEG-L-asparaginase,
microtubule polymer stabilizers such as the taxanes paclitaxel and docetaxel, topisomerase I inhibitors such as the camptothecins irinotecan and topotecan, and topoisomerase II inhibitors such as podophyllotoxin, amsacrine, etoposide, teniposide, losoxantrone and actinomycin;
hormones and hormone antagonists, including androgens such as fluoxymesterone and testolactone,
antiandrogens such as bicalutamide, cyproterone, flutamide, and nilutamide, corticosteroids such as dexamethasone and prednisone,
aromatase inhibitors such as aminoglutethimide, anastrozole, exemestane, formestane, and letrozole,
estrogens such as diethylstilbestrol,
antiestrogens such as fulvestrant, raloxifene, tamoxifen, and toremifine, luteinising hormone-releasing hormone (LHRH) agonists and antagonists such as abarelix, buserelin, goserelin, leuprolide, histrelin, desorelin, nafarelin acetate and triptorelin,
progestins such as medroxyprogesterone acetate and megestrol acetate, and thyroid hormones such as levothyroxine and liothyronine;
PKB pathway inhibitors, including perifosine, enzastaurin hydrochloride, and triciribine,
P13K inhibitors such as semaphore and SF1126, and
MTOR inhibitors such as rapamycin and analogues;
CDK inhibitors, including seliciclib, alvocidib, and 7-hydroxystaurosporine;
COX-2 inhibitors, including celecoxib;
HDAC inhibitors, including trichostatin A, suberoylanilide hydroxamic acid, and chlamydocin;
DNA methylase inhibitors, including temozolomide; and
miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer, and proteasome inhibitors such as bortezomib.

Molecular targeted therapy agents including:

functional therapeutic agents, including gene therapy agents,
antisense therapy agents,
tyrosine kinase inhibitors such as erlotinib hydrochloride, gefitinib, imatinib mesylate, and semaxanib,
Raf inhibitors such as sorafenib, and
gene expression modulators such as the retinoids and rexinoids, for example adapalene, bexarotene, trans-retinoic acid, 9-cis-retinoic acid, and N-(4-hydroxyphenyl)retinamide; and
phenotype-directed therapy agents, including monoclonal antibodies such as alemtuzumab, bevacizumab, cetuximab, ibritumomab tiuxetan, rituximab, and trastuzumab, immunotoxins such as gemtuzumab ozogamicin, radioimmunoconjugates such as I-tositumobab, and
cancer vaccines.
Biologic therapy agents including:
interferons such as interferon-[alpha]2a and interferon-[alpha]2b, and interleukins such as aldesleukin, denileukin diftitox, and oprelvekin. Axl inhibiting agents including 1-(6,7-dihydro-5H-benzo[6,7]cyclohepta[1,2-c]pyridazin-3-yl)-N3-47-(S)-pyrrolidin-1-yl)-6,7,8,9-tetrahydro-5H-benzo[7]annulene-2-yl)-1H-1,2,4-triazole-3,5-diamine (BGB324/R428), CH5451098 (Roche) and Axl inhibitors described in PCT/US07/089177, PCT/US2010/021275 and PCT/EP2011/004451, incorporated herein by reference.

In addition to these agents intended to act against cancer cells, anticancer therapies include the use of protective or adjunctive agents, including:

cytoprotective agents such as amifostine, and dexrazoxane,
phosphonates such as pamidronate and zoledronic acid, and
stimulating factors such as epoetin, darbeopetin, filgrastim, PEG-filgrastim, and sargramostim.

Many combination chemotherapeutic regimens are known to the art, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin, and the like.

According to a further aspect of the invention is provided a method of selecting patients, preferably human patients, for treatment of an Axl-related condition, the method comprising identifying patients having elevated Slfn11 activity or expression and selecting thus identified patients for treatment. Patients may be identified according to the methods of the invention as described herein.

Preferably the Axl-related condition is cancer. The cancer may be one or more of the following cancers: Leukemias such as but not limited to acute myelocytic leukemias (AMLs) such as myeloblastic, promyelocytic, myelomonocytic, monocytic, erythroleukemia leukemias and myelodysplastic syndrome, acute leukemia, acute lymphocytic leukemia, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; genital cancers such as penile cancer; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendothel iosarcoma, mesothelioma, synovioma, hem angioblastoma, epithelial carcinoma, cystadenocarcinoma, bronchogenic carcinoma, sweat gland carcinoma, sebaceous gland carcinoma, papillary carcinoma and papillary adenocarcinomas. Preferably, the cancer is selected from acute myelocytic leukemia (AML), breast, melanoma, prostate, ovarian, colorectal, lung or glioma cancer; the cancer may be metastatic. More preferably the cancer is acute myelocytic leukemias (AMLs).

The treatment of metastatic cancer depends on where the primary tumor is located. When breast cancer spreads to the lungs, for example, it remains a breast cancer and the treatment is determined by the metastatic cancer origin within the breast, not by the fact that it is now in the lung. About 5 percent of the time, metastatic cancer is discovered but the primary tumor cannot be identified. The treatment of these metastatic cancers is dictated by their location rather than their origin. Metastatic cancers are named by the tissue of the original tumor (if known). For example, a breast cancer that has spread to the brain is called metastatic breast cancer to the brain. Patients identified or selected according to the methods of the invention may be treated, or selected for treatment. For example, if Slfn11 expression is shown to be upregulated in a primary tumor, this can be used to infer an increased probability of metastasis. This information can be used as a guide to treatment options, i.e. more aggressive anti-cancer surgical, chemotherapeutic or radiotherapeutic treatment such as radical mastectomy. In some embodiments, treatment comprises administration of an Akt3 and/or Axl inhibitor, optionally in combination with a further therapeutic agent described herein or known in the art. Preferably the Axl inhibitor is BGB324/R428.

The invention also provides a method of identifying a compound which inhibits Axl activity, a method comprising contacting a cell from a cell line according to the invention with a test compound and determining inhibition of Slfn11 expression or activity in the cell.

One aspect of the invention relates to the use of Slfn11 as a biomarker for detecting the occurrence of epithelial-to-mesenchymal transition (EMT) in a subject. In some embodiments, an increase in the expression and/or activation of Slfn11 is indicative of the occurrence of epithelial-to-mesenchymal transition (EMT).

Metastasis to distant sites is the most common cause of death from solid tumors (Gupta 2006, Sporn 1996). To accomplish this, tumor cells discard epithelial restraints, redefine junctional complexes and acquire invasive motility to break across the basement membrane border. These metastatic cells then intravasate into the lymphatic and hematogenous circulation, disseminating to distant sites in the body. A few of these metastatic cells succeed in extravasating through the capillary wall and in rare cases colonize the foreign tissue stroma (Weinberg et al). This malignant process is facilitated by an epithelial-to-mesenchymal transition (EMT), a developmental program where epithelial cells transiently assume a mesenchymal phenotype during gastrulation and organogenesis, allowing single cell invasive movement away from the epithelial layer (Hall, 1985; Thierry, 2002). The EMT program is initiated by contextual activation of morphogen signaling pathways that induce the expression of transcriptional regulators, including Twist, Snail, Slug and Zeb2, which alter the expression of junctional complex proteins (Thiery and SLeeman 2006). The EMT gene expression profile reflects the phenotypic shift, repression of E-cadherin and cytokeratins with induction of vimentin and N-cadherin (Weinberg et al 2007).

The term “marker” or “biomarker” is used herein to refer to a gene or protein whose expression in a sample derived from a cell or mammal is altered or modulated, for example, up or down regulated, when epithelial-to-mesenchymal transition (EMT) takes place. Where the biomarker is a protein, modulation or alteration of expression encompasses modulation through different post-translational modifications.

Post-translational modifications are covalent processing events that change the properties of a protein by proteolytic cleavage or by addition of a modifying group to one or more amino acids. Common post-translational modifications include phosphorylation, acetylation, methylation, acylation, glycosylation, GPI anchor, ubiquitination and so forth. A review of such modifications and methods for detection may be found in Mann et al. Nature Biotechnology March 2003, Vol. 21, pages 255-261.

Also provided herein is the use of Slfn11 as a biomarker for detecting the expression and/or activation of Axl, wherein an increase in the expression and/or activation of Slfn11 is indicative of an increase in the expression and/or activation of Axl.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

Detection of the level of expression including gene expression may be performed by any one of the methods known in the art, particularly by microarray analysis, Western blotting or by PCR techniques such as QPCR. Altered expression may also be detected by analysing protein content of samples using methods such as ELISA, PET or SELDI-TOF MS as described herein and using further analytical techniques such as 2Dgel electrophoresis. Techniques such as this can be particularly useful for detecting altered expression in the form of alternative post translationally modified forms of a protein.

Suitable samples include, but are not limited to, tissue samples such as biopsy, blood, urine, buccal scrapes etc, serum, plasma or tissue culture supernatant samples. In one embodiment, gene expression is preferably detected in tumour cells, particularly cells derived from a tumour such as breast, lung, gastric, head and neck, colorectal, renal, pancreatic, uterine, hepatic, bladder, endometrial and prostate cancers and leukemias or from blood cells such as lymphocytes and, preferably, PBMCs such as lymphocytes.

In detection of proteins in serum and, in particular, in plasma samples of patients, samples are removed and subjected to protein analytical techniques such as flow cytometry, mass cytometry (CyTOF), ELISA, PET and SELDI-TOF MS.

In one preferred embodiment, the method comprises extracting RNA from said sample and detecting gene expression by QPCR.

In one embodiment, gene expression is detected by detecting protein products such as, for example, by Western Blot.

A further aspect of the invention provides a method for detecting the occurrence of epithelial-to-mesenchymal transition (EMT) in a sample, said method comprising determining the expression level or activation of Slfn11 in a sample isolated from a cell, group of cells, an animal model or human as compared to a control sample, wherein an increase in the expression level or activation of Slfn11 relative to the control sample is indicative of the occurrence of epithelial-to-mesenchymal transition (EMT).

A further aspect of the invention relates to a method for identifying an agent capable of inhibiting or reversing epithelial-to-mesenchymal transition (EMT), said method comprising administering said agent to a cell, group of cells or animal model, and monitoring the activation and/or the expression of Slfn11.

In one embodiment, the method comprises:

(i) administering the agent to a cell, group of cells or an animal model, not a human; and
(ii) measuring Slfn11 expression and/or Slfn11 activation in samples derived from the treated and the untreated cells or animal model; and
(iii) detecting a decrease in the expression and/or activation of Slfn11 in the treated sample as compared to the untreated sample as an indication of the ability to inhibit or reverse epithelial-to-mesenchymal transition (EMT).

In some embodiments, the animal model is not a human.

In some embodiments, the level of expression of Slfn11 is assessed by determining the copy number of the gene encoding Slfn11 relative to a control sample, wherein an increase in the copy number indicates an increased level of expression of Slfn11.

In some embodiments, the level of expression of Slfn11 is assessed by determining the level of Slfn11 protein or mRNA.

### Slfn11

Human Slfn11 (Schlafen family member 11) is described in UniProt entry Q7Z7L1 The “canonical” sequence (Q7Z7L1-1) is as follows:

SEQ ID NO: 1
MEANQCPLVVEPSYPDLVINVGEVTLGEENRKKLQKIQRDQEKERVMRAA
CALLNSGGGVIRMAKKVEHPVEMGLDLEQSLRELIQSSDLQAFFETKQQG
RCFYIFVKSWSSGPFPEDRSVKPRLCSLSSSLYRRSETSVRSMDSREAFC
FLKTKRKPKILEEGPFHKIHKGVYQELPNSDPADPNSDPADLIFQKDYLE
YGEILPFPESQLVEFKQFSTKHFQEYVKRTIPEYVPAFANTGGGYLFIGV
DDKSREVLGCAKENVDPDSLRRKIEQAIYKLPCVHFCQPQRPITFTLKIV
NVLKRGELYGYACMIRVNPFCCAVFSEAPNSWIVEDKYVCSLTTEKWVGM
MTDTDPDLLQLSEDFECQLSLSSGPPLSRPVYSKKGLEHKKELQQLLFSV
PPGYLRYTPESLWRDLISEHRGLEELINKQMQPFFRGILIFSRSWAVDLN
LQEKPGVICDALLIAQNSTPILYTILREQDAEGQDYCTRTAFTLKQKLVN
MGGYTGKVCVRAKVLCLSPESSAEALEAAVSPMDYPASYSLAGTQHMEAL
LQSLVIVLLGFRSLLSDQLGCEVLNLLTAQQYEIFSRSLRKNRELFVHGL
PGSGKTIMAMKIMEKIRNVFHCEAHRILYVCENQPLRNFISDRNICRAET
RKTFLRENFEHIQHIVIDEAQNFRTEDGDWYGKAKSITRRAKGGPGILWI
FLDYFQTSHLDCSGLPPLSDQYPREELTRIVRNADPIAKYLQKEMQVIRS
NPSFNIPTGCLEVFPEAEWSQGVQGTLRIKKYLTVEQIMTCVADTCRRFF
DRGYSPKDVAVLVSTAKEVEHYKYELLKAMRKKRVVQLSDACDMLGDHIV
LDSVRRFSGLERSIVFGIHPRTADPAILPNVLICLASRAKQHLYIFPWGG
H

mRNA encoding Slfn11 (AK074184.1) has sequence as follows:

SEQ ID NO: 2
GTGCAGTGGCACGATCTTGGTTCACCACAATCTCGTCTCGAAGGCTCAGG
TGATTCTCTCACCTCAGCCGCCTGAGTAGCTGGGACCACAGTTTCAGCTG
TGAGTTCAACATGGAGGCAAATCAGTGCCCCCTGGTTGTGGAACCATCTT
ACCCAGACCTGGTCATCAATGTAGGAGAAGTGACTCTTGGAGAAGAAAAC
AGAAAAAAGCTGCAGAAAATTCAGAGAGACCAAGAGAAGGAGAGAGTTAT
GCGGGCTGCATGTGCTTTATTAAACTCAGGAGGAGGAGTGATTCGAATGG
CCAAGAAGGTTGAGCATCCCGTGGAGATGGGACTGGATTTAGAACAGTCT
TTGAGAGAGCTTATTCAGTCTTCAGATCTGCAGGCTTTCTTTGAGACCAA
GCAACAAGGAAGGTGTTTTTACATTTTTGTTAAATCTTGGAGCAGTGGCC
CTTTCCCTGAAGATCGCTCTTTCAAGCCCCGCCTTTGCAGCCTCAGTTCT
TCATTATACCTGTAGATCTGAGACCTCTGTGCGTTCCATGGACTCAAGAG
AGGCATTCTGTTTCCTGAAGACCAAAAGGAAGCCAAAAATCTTGGAAGAA
GGACCTTTTCACAAAATTCACAAGGGTGTATACCAAGAGCTCCCTAACTC
GGATCCTGCTGACCCAAACTCGGATCCTGCTGACCTAATTTTCCAAAAAG
ACTATCTTGAATATGGTGAAATCCTGCCTTTTCCTGAGTCTCAGTTAGTA
GAGTTTAAACAGTTCTCTACAAAACACTTCCAAGAATATGTAAAAAGGAC
AATTCCAGAATACGTCCCTGCATTTGCAAACACTGGAGGAGGCTATCTTT
TTATTGGAGTGGATGATAAGAGTAGGGAAGTCCTGGGATGTGCAAAAGAA
AATGTTGACCCTGACTCTTTGAGAAGGAAAATAGAACAAGCCATATACAA
ACTACCTTGTGTTCATTTTTGCCAACCCCAACGCCCGATAACCTTCACAC
TCAAAATTGTGGATGTGTTAAAAAGGGGAGAGCTCTATGGCTATGCTTGC
ATGATCAGAGTAAATCCCTTCTGCTGTGCAGTGTTCTCAGAAGCTCCCAA
TTCATGGATAGTGGAGGACAAGTACGTCTGCAGCCTGACAACCGAGAAAT
GGGTAGGCATGATGACAGACACAGATCCAGATCTTCTACAGTTGTCTGAA
GATTTTGAATGTCAGCTGAGTCTATCTAGTGGGCCTCCCCTTAGCAGACC
AGTGTACTCCAAGAAAGGCCTGGAACATAAAAAGGAACTCCAGCAACTTT
TATTTTCAGTCCCACCAGGATATTTGCGATATACTCCAGAGTCACTCTGG
AGGGACCTGATCTCAGAGCACAGAGGACTAGAGGAGTTAATAAATAAGCA
AATGCAACCTTTCTTTCGGGGAATTTTGATCTTCTCTAGAAGTTGGGCTG
TGGACCTGAACTTGCAGGAGAAGCCAGGAGTCATCTGTGATGCTCTGCTG
ATAGCACAGAACAGCACCCCCATTCTCTACACCATTCTCAGGGAGCAGGA
TGCAGAGGGCCAGGACTACTGCACTCGCACTGCCTTTACTTTGAAGCAGA
AGCTAGTGAACATGGGGGGCTACACCGGGAAGGTGTGTGTCAGGGCCAAG
GTCCTCTGCCTGAGTCCTGAGAGCAGCGCAGAGGCCTTGGAGGCTGCAGT
GTCTCCGATGGATTACCCTGCGTCCTATAGCCTTGCAGGCACCCAGCACA
TGGAAGCCCTGCTGCAGTCCCTCGTGATTGTCTTACTCGGCTTCAGGTCT
CTCTTGAGTGACCAGCTCGGCTGTGAGGTTTTAAATCTGCTCACAGCCCA
GCAGTATGAGATATTCTCCAGAAGCCTCCGCAAGAACAGAGAGTTGTTTG
TCCACGGCTTACCTGGCTCAGGGAAGACCATCATGGCCATGAAGATCATG
GAGAAGATCAGGAATGTGTTTCACTGTGAGGCACACAGAATTCTCTACGT
TTGTGAAAACCAGCCTCTGAGGAACTTTATCAGTGATAGAAATATCTGCC
GAGCAGAGACCCGGAAAACTTTCCTAAGAGAAAACTTTGAACACATTCAA
CACATCGTCATTGACGAAGCTCAGAATTTCCGTACTGAAGATGGGGACTG
GTATGGGAAGGCAAAAAGCATCACTCGGAGAGCAAAGGGTGGCCCAGGAA
TTCTCTGGATCTTTCTGGATTACTTTCAGACCAGCCACTTGGATTGCAGT
GGCCTCCCTCCTCTCTCAGACCAATATCCAAGAGAAGAGCTCACCAGAAT
AGTTCGCAATGCAGATCCAATAGCCAAGTACTTACAAAAAGAAATGCAAG
TAATTAGAAGTAATCCTTCATTTAACATCCCCACTGGGTGCCTCGAGGTA
TTTCCTGAAGCCGAATGGTCCCAGGGTGTTCAGGGAACCTTACGAATTAA
GAAATACTTGACTGTGGAGCAAATAATGACCTGTGTGGCAGACACGTGCA
GGCGCTTCTTTGATAGGGGCTATTCTCCAAAGGATGTTGCTGTGCTTGTC
AGCACCGCAAAAGAAGTGGAGCACTATAAGTATGAGCTCTTGAAAGCAAT
GAGGAAGAAAAGGGTGGTGCAGCTCAGTGATGCATGTGATATGTTGGGTG
ATCACATTGTGTTGGACAGTGTTCGGCGATTCTCAGGCCTGGAAAGGAGC
ATAGTGTTTGGGATCCATCCAAGGACAGCTGACCCAGCTATCTTACCCAA
TGTTCTGATCTGTCTGGCTTCCAGGGCAAAACAACACCTGTATATTTTTC
CGTGGGGTGGCCATTAGGAAGAACTCCAAATCAAAATGCTATGTAAATGT
CTATGGGTGACAGTCTGCTGATGGTAGAAACCTTTCTTTTTAGTTCACAA
GTCAGAGATTTGGACGGAGCTGACACAAAGAGTTTGGAGCTCCCCCATTT
CTGGCTCTCCTTTCAGGGGTTCCTTCCCCAACCCTTTTCAGCAGCGGTGG
CTGCCCCCCATTCTGACCCCTGACTCTTCCAGCCAGAAAGATGGTGGTTT
TCTAAAGGAACTTTAGCTGTCCTGCACAATGCCGATCTGTGTCTTGCATT
TTGGGTAAAAGCCATAAAAATAAGAAACTCAGCCTGTGGCCTTTCTTTCT
TCCAAGGCTGGGCTTCTTTTTTTAAGTGACTTCATGCAGTTTGTTGCTTT
TAAAAATTTGTCCAGAATCGTTTTCTGCAGAAGCATGGTCTGTTAGGAGC
TTACTGGCCGTAGCAGAAGCAATTGTTTCCTGAATTCTTGACATTTATCT
TTGCTGTATTCATTTAGGGCTTGGGAGAGTCCGAAGATAATTCAGTCACT
GTCAGATTAATAATTCTGTCAGGACAAAGAATACCGTTATGATTATTTAA
TCCTTTAAAATTGTGGTCTCCAGAGCTTGTTCTCAGAATGGCCCAGACCA
AGCCTTAATTGTGATAGTGAATATTAATGGTCACTTTAAGGAGAAATTAT
AGGCCAAGATGAAATGAACATAAACCTGTTTGCCCTGGCTTTCAGTGGAA
GATGATATTAGAGACCAAAATCTGGTTCTGAAGGTGTGTATCAGCCCTAA
GGTGAACCAGACTTGGGAAAGATTGTCTTTAAAAATCAATGAGTTTATGT
TTTAACTTCTCAGCTTAGTTCTATGCATTGCTCTATAACACACCTAGTTA
AGTTTTATGTTATTCTTGAACTGTGATTTTTTTTCTATTTACTTTCATGG
TTTGGTGGGCCATTGTTATGGACTGAATGTTTGTGTCCCACCCTTCACCC
CCAAATTCCCGTGTTGAAGCCCCAACCTGCACTGTGGAGCTGGGGCTGCT
AAGGAAGTAATTAAGGTTACATGAAGTCATGGTGGGGCTCTGATCTGCTA
AGGTTGGTGTCCTTATAGGGAGAGACCCCAGAGAGCTTGTTCCCTCCCTC
CCTGTGCATGCAAACAAGAGGGCATGGGAGCACACAGAGAGATGGCAGCC
ACCTACAAGCCAAGAGGAGAAGCCTCACAATCAAACTCTCGCTGCTGGCG
AGAGTCTTGGACTCTGTCTTGGACTTCCAGCCTCCAGACTGTGAGAAACA
AATTTCTGTTGTTTCAGCTTCTCAGTCTCTGGTGTTTTGTTATTGCAGCC
TGAGAACACAGCTGTACGATTATTTGTCAAACAGAAAACACTGATACTTA
ACAATGCTAATGCAATTATTTATTTGCTTTTCAGTCTCTACAAAACGTTC
TAAAACACTAATCTAAATATTAACAGTAAAATATTTGCATAACTAATGGA
AACTAAGAAATCATATGACCAATATTTCACTTATTGGTAATCTTACTCTA
CTGATTTCCCCCCAGACTGTGATTTTTGAACTTCCTTGCCTTTCTCCTGT
CTTTCTGTGTTTATTCATGGAATTCCAGTTATCTGGGCTTGAAATTGCAG
GCTCTCCTAACTTAAGCAAAATCTGACAGATCAGCAAAATGAGATAAATG
TTTCTTTTTTCTTTCTGACTGCATTAAATCAGATACAACTCAGCATTAAA
AAGCTATCTTTGTAAATGTTGTTACTAATAAATTAGTCTTATAAGATCCC
TGGACTTTGGAGTTGTTGCAATGTCTTTGAGAGTAATTCTTTAAAAGTCT
AATTTCGACTGGTTGTATCTCTTTATGATTTATTGCCCCACTAACAACAT
TTGAAACAATATAATATTTTAAAATGTATAAATAATTATGAATTTTTGTT
TAGAACAAAGAGGATTACTGATATTTGTTTCCCTATGAATGGCAAAAGGT
TTAGCTTACTACTGCATTTCTGTTTTAAATAAAAAGTTGAGAGTTTGTGT
CTCATTAAACTG

### Measuring Altered Expression of Gene/Protein Markers

Levels of gene and protein expression may be determined using a number of different techniques.

### (a) At the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

For Northern blotting, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

Nuclease Protection Assays (including both ribonuclease protection assays and 51 nuclease assays) provide an extremely sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. NPAs allow the simultaneous detection of several RNA species.

In situ hybridization (ISH) is a powerful and versatile tool for the localization of specific mRNAs in cells or tissues. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. Alternatively, tissue can be sectioned frozen and post-fixed in paraformaldehyde. After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents. This latter method of detection is the basis for Fluorescent In Situ Hybridisation (FISH).

Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection. Relative quantitative RT-PCR involves amplifying an internal control simultaneously with the gene of interest. The internal control is used to normalize the samples. Once normalized, direct comparisons of relative abundance of a specific mRNA can be made across the samples. Commonly used internal controls include, for example, GAPDH, HPRT, actin and cyclophilin. Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification (TAS) methods have been described in the literature, for example, general reviews of these methods in Landegren, U. et al., Science 242:229-237 (1988) and Lewis, R., Genetic Engineering News 10:1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., 1994, Gynaecologic Oncology 52:247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990, Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B., 1989, Genomics 4:560. In the Qβ Replicase technique, RNA replicase for the bacteriophage Qβ, which replicates single-stranded RNA, is used to amplify the target DNA, as described by Lizardi et al., 1988, Bio/Technology 6:1197.

Quantitative PCR (Q-PCR) is a technique which allows relative amounts of transcripts within a sample to be determined. A suitable method for performing QPCR is described herein.

Alternative amplification technology can be exploited in the present invention. For example, rolling circle amplification (Lizardi et al., 1998, Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., 1992, Proc. Natl. Acad. Sci. USA 80:392) begins with a specifically defined sequence unique to a specific target.

Suitable probes for detecting the expression of Slfn11 identified herein may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like. Suitable kits may comprise, for example, primers for a QPCR reaction or labelled probes for performing FISH.

### (b) At the Polypeptide Level

Altered gene or protein expression may also be detected by measuring the polypeptides encoded by the Slfn11 gene. This may be achieved by using molecules which bind to the polypeptides encoded by the Slfn11 gene. Suitable molecules/agents which bind either directly or indirectly to the polypeptides in order to detect the presence of the protein include naturally occurring molecules such as peptides and proteins, for example antibodies, or they may be synthetic molecules.

Antibodies for the Slfn11 gene or protein may be derived from commercial sources or through techniques which are familiar to those skilled in the art. In one embodiment, and where altered expression manifests itself through the expression of alteration of post translationally-modified forms of a protein biomarker, antibodies specific for those different forms may be used.

Methods for production of antibodies are known by those skilled in the art. If polyclonal antibodies are desired, a selected mammal (e.g., mouse, rabbit, goat, horse, etc.) is immunised with an immunogenic polypeptide bearing an epitope(s) from a polypeptide. Serum from the immunised animal is collected and treated according to known procedures. If serum containing polyclonal antibodies to an epitope from a polypeptide contains antibodies to other antigens, the polyclonal antibodies can be purified by immunoaffinity chromatography. Techniques for producing and processing polyclonal antisera are known in the art. In order to generate a larger immunogenic response, polypeptides or fragments thereof may be haptenised to another polypeptide for use as immunogens in animals or humans.

Monoclonal antibodies directed against epitopes in polypeptides can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes in the polypeptides of the invention can be screened for various properties; i.e., for isotype and epitope affinity. An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes whole antibodies, or fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP239400A. For example: monoclonal and polyclonal antibodies, recombinant antibodies, proteolytic and recombinant fragments of antibodies (Fab, Fv, scFv, diabodies), single-domain antibodies (VHH, sdAb, nanobodies, IgNAR, VNAR), and proteins unrelated to antibodies, which have been engineered to have antibody-like specific binding, such as the following:

| Name | Based on: | |
|---|---|---|
| Affibodies | Protein A, Z domain | 6 kDa |
| Affitins | Sac7d (from Sulfolobus acidocaldarius) | 7 kDa |
| Anticalins | Lipocalins | 20 kDa |
| DARPins | Ankyrin repeat motif | 14 kDa |
| Fynomers | Fyn, SH3 domain | 7 kDa |
| Kunitz domain | Various protease inhibitors | 6 kDa |
| peptides | | |
| Monobodies | Fibronectin | |

Standard laboratory techniques such as immunoblotting as described above can be used to detect altered levels of Slfn11 activity, as compared with untreated cells in the same cell population.

Gene expression may also be determined by detecting changes in post-translational processing of polypeptides or post-transcriptional modification of nucleic acids. For example, differential phosphorylation of polypeptides, the cleavage of polypeptides or alternative splicing of RNA, and the like may be measured. Levels of expression of gene products such as polypeptides, as well as their post-translational modification, may be detected using proprietary protein assays or techniques such as 2D polyacrylamide gel electrophoresis.

Antibodies may be used for detecting Slfn11 expression by a method which comprises: (a) providing an antibody; (b) incubating a biological sample with said antibody under conditions which allow for the formation of an antibody-antigen complex; and (c) determining whether antibody-antigen complex comprising said antibody is formed.

Suitable samples include extracts of tissues such as brain, breast, ovary, lung, colon, pancreas, testes, liver, muscle and bone tissues or from neoplastic growths derived from such tissues. Other suitable examples include blood or urine samples.

Antibodies that specifically bind to Slfn11 proteins can be used in diagnostic or prognostic methods and kits that are well known to those of ordinary skill in the art to detect or quantify the expression of Slfn11 protein in a body fluid or tissue. Results from these tests can be used to diagnose or predict the occurrence or recurrence of cancer and other cell motility or cell survival-mediated diseases, or to assess the effectiveness of drug dosage and treatment.

Antibodies can be assayed for immunospecific binding by any method known in the art. The immunoassays which can be used include but are not limited to competitive and non-competitive assay systems using techniques such as western blots, immunohistochemistry, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety).

Antibodies for use in the invention are preferably bound to a solid support and/or packaged into kits in a suitable container along with suitable reagents, controls, instructions and the like.

Other methods include, but are not limited to, 2D-PAGE although this is less suitable for large-scale screening. Newer techniques include matrix-assisted laser desorption ionization time of flight mass spectrometry (MALDI-TOF MS). In MALDI-TOF analysis, proteins in a complex mixture are affixed to a solid metallic matrix, desorbed with a pulsed laser beam to generate gas-phase ions that traverse a field-free flight tube, and are then separated according to their mass-dependent velocities. Individual proteins and peptides can be identified through the use of informatics tools to search protein and peptide sequence databases. Surface-enhanced laser desorption/ionisation time of flight MS (SELDI-TOF MS) is an affinity-based MS method in which proteins are selectively adsorbed to a chemically modified solid surface, impurities are removed by washing, an energy-absorbing matrix is applied, and the proteins are identified by laser desorption mass analysis.

SELDI-TOF-MS can be used for the detection of the appearance/loss of either intact proteins or fragments of specific proteins. In addition SELDI-TOF-MS can also be used for detection of post translational modifications of proteins due to the difference in mass caused by the addition/removal of chemical groups. Thus phosphorylation of a single residue will cause a mass shift of 80 Da due to the phosphate group. A data base of molecular weights that can be attributed to post-translational modifications is freely accessible on the internet (http://www.abrf.org/index.cfm/dm.home?avgmass=all). Moreover specific polypeptides can be captured by affinity-based approaches using SELDI-TOF-MS by employing antibodies that specifically recognise a post-translationally modified form of the protein, or that can recognise all forms of the protein equally well.

### Arrays

Array technology and the various techniques and applications associated with it is described generally in numerous textbooks and documents. These include Lemieux et al., 1998, Molecular Breeding 4:277-289; Schena and Davis. Parallel Analysis with Biological Chips. in PCR Methods Manual (eds. M. Innis, D. Gelfand, J. Sninsky); Schena and Davis, 1999, Genes, Genomes and Chips. In DNA Microarrays: A Practical Approach (ed. M. Schena), Oxford University Press, Oxford, U K, 1999); The Chipping Forecast (Nature Genetics special issue; January 1999 Supplement); Mark Schena (Ed.), Microarray Biochip Technology, (Eaton Publishing Company); Cortes, 2000, The Scientist 14(17):25; Gwynne and Page, Microarray analysis: the next revolution in molecular biology, Science, 1999, August 6; Eakins and Chu, 1999, Trends in Biotechnology, 17:217-218, and also at various world wide web sites.

Array technology overcomes the disadvantages with traditional methods in molecular biology, which generally work on a “one gene in one experiment” basis, resulting in low throughput and the inability to appreciate the “whole picture” of gene function. Currently, the major applications for array technology include the identification of sequence (gene/gene mutation) and the determination of expression level (abundance) of genes. Gene expression profiling may make use of array technology, optionally in combination with proteomics techniques (Celis et al., 2000, FEBS Lett, 480(1):2-16; Lockhart and Winzeler, 2000, Nature 405(6788):827-836; Khan et al., 1999, 20(2):223-9). Other applications of array technology are also known in the art; for example, gene discovery, cancer research (Marx, 2000, Science 289: 1670-1672; Scherf et alet al., 2000, Nat Genet 24(3):236-44; Ross et al., 2000, Nat Genet 2000, 24(3):227-35), SNP analysis (Wang et al., 1998, Science 280(5366):1077-82), drug discovery, pharmacogenomics, disease diagnosis (for example, utilising microfluidics devices: Chemical & Engineering News, Feb. 22, 1999, 77(8):27-36), toxicology (Rockett and Dix (2000), Xenobiotica 30(2):155-77; Afshari et al., 1999, Cancer Res 59(19):4759-60) and toxicogenomics (a hybrid of functional genomics and molecular toxicology). The goal of toxicogenomics is to find correlations between toxic responses to toxicants and changes in the genetic profiles of the objects exposed to such toxicants (Nuwaysir et al., 1999, Molecular Carcinogenesis 24:153-159).

In the context of the present invention, array technology can be used, for example, in the analysis of the expression of Slfn11 protein or mRNA. In one embodiment, array technology may be used to assay the effect of a candidate compound on Slfn11 activity.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array.

The samples (e.g., members of a library) are generally fixed or immobilised onto a solid phase, preferably a solid substrate, to limit diffusion and admixing of the samples. In a preferred embodiment, libraries of DNA binding ligands may be prepared. In particular, the libraries may be immobilised to a substantially planar solid phase, including membranes and non-porous substrates such as plastic and glass. Furthermore, the samples are preferably arranged in such a way that indexing (i.e., reference or access to a particular sample) is facilitated. Typically the samples are applied as spots in a grid formation. Common assay systems may be adapted for this purpose. For example, an array may be immobilised on the surface of a microplate, either with multiple samples in a well, or with a single sample in each well. Furthermore, the solid substrate may be a membrane, such as a nitrocellulose or nylon membrane (for example, membranes used in blotting experiments). Alternative substrates include glass, or silica based substrates. Thus, the samples are immobilised by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilised to arrange and fix the samples on the chip.

The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14(11):26.

Techniques for producing immobilised libraries of DNA molecules have been described in the art. Generally, most prior art methods described how to synthesise single-stranded nucleic acid molecule libraries, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produced the immobilised DNA libraries of the present invention. U.S. Pat. No. 5,837,832 also provides references for earlier techniques that may also be used.

Arrays of peptides (or peptidomimetics) may also be synthesised on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a target or probe) and reactive library members occur is determined, thereby identifying the sequences of the reactive library members on the basis of spatial location. These methods are described in U.S. Pat. No. 5,143,854; WO 90/15070 and WO 92/10092; Fodor et al., 1991, Science 251:767; Dower and Fodor, 1991, Ann. Rep. Med. Chem. 26:271.

To aid detection, targets and probes may be labelled with any readily detectable reporter, for example, a fluorescent, bioluminescent, phosphorescent, radioactive, etc reporter. Such reporters, their detection, coupling to targets/probes, etc are discussed elsewhere in this document. Labelling of probes and targets is also disclosed in Shalon et al., 1996, Genome Res 6(7):639-45.

Specific examples of DNA arrays include the following:

Format I: probe cDNA (˜500-˜5,000 bases long) is immobilized to a solid surface such as glass using robot spotting and exposed to a set of targets either separately or in a mixture. This method is widely considered as having been developed at Stanford University (Ekins and Chu, 1999, Trends in Biotechnology, 17:217-218).

Format II: an array of oligonucleotide (˜20-˜25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. Such a DNA chip is sold by Affymetrix, Inc., under the GeneChip® trademark. Examples of some commercially available microarray formats are set out, for example, in Marshall and Hodgson, 1998, Nature Biotechnology 16(1):27-31.

Data analysis is also an important part of an experiment involving arrays. The raw data from a microarray experiment typically are images, which need to be transformed into gene expression matrices—tables where rows represent for example genes, columns represent for example various samples such as tissues or experimental conditions, and numbers in each cell for example characterize the expression level of the particular gene in the particular sample. These matrices have to be analyzed further, if any knowledge about the underlying biological processes is to be extracted. Methods of data analysis (including supervised and unsupervised data analysis as well as bioinformatics approaches) are disclosed in Brazma and Vilo J, 2000, FEBS Lett 480(1):17-24.

As disclosed above, proteins, polypeptides, etc may also be immobilised in arrays. For example, antibodies have been used in microarray analysis of the proteome using protein chips (Borrebaeck CA, 2000, Immunol Today 21(8):379-82). Polypeptide arrays are reviewed in, for example, MacBeath and Schreiber, 2000, Science, 289(5485):1760-1763.

### Pharmaceutical Composition

A further aspect relates to a pharmaceutical composition comprising an Slfn11 modulator, such as an activator or inhibitor, or other agent identified according to any of the above-described methods admixed with a pharmaceutically acceptable diluent, excipient or carrier.

For use according to the present invention, the agent may be presented as a pharmaceutical formulation, comprising the compounds or physiologically acceptable salt, ester or other physiologically functional derivative thereof, together with one or more pharmaceutically acceptable carriers and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients”, 2nd Edition, (1994), Edited by A Wade and PJ Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p hydroxybenzoic acid. Antioxidants and suspending agents may be also used.

Pharmaceutical formulations include those suitable for oral, topical (including den ial, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose fot mulations such as boluses, capsules or tablets each containing a predetermined amount of active agent. A tablet may be made by compression or moulding, optionally with one or more accessory ingredients. Compressed tablets may be prepared by compressing in a suitable machine an active agent in a free-flowing form such as a powder or granules optionally mixed with a binder, lubricant, inert diluent, lubricating agent, surface-active agent or dispersing agent. Moulded tablets may be made by moulding an active agent with an inert liquid diluent. Tablets may be optionally coated and, if uncoated, may optionally be scored. Capsules may be prepared by filling an active agent, either alone or in admixture with one or more accessory ingredients, into the capsule shells and then sealing them in the usual manner. Cachets are analogous to capsules wherein an active agent together with any accessory ingredient(s) is sealed in a rice paper envelope. An active agent may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active agent is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active agent with the softened or melted carrier(s) followed by chilling and shaping in moulds.

Pharmaceutical formulations suitable for parenteral administration include sterile solutions or suspensions of an active agent in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active agent may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active agent, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active agent is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microliters, upon each operation thereof.

As a further possibility an active agent may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active agent in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing an agent into association with a pharmaceutically or veterinarily acceptable carrier or vehicle.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier. Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In accordance with this invention, an effective amount of agent may be administered to inhibit Slfn11. Of course, this dosage amount will further be modified according to the type of administration of the agent. For example, to achieve an “effective amount” for acute therapy, parenteral administration is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit a kinase. The agents may be administered one to four times daily at a level to achieve a total daily dose of about 0.4 to about 400 mg/kg/day. The precise amount of an active-agent which is therapeutically effective, and the route by which such agent is best administered, is readily determined by one of ordinary skill in the art by comparing the blood level of the agent to the concentration required to have a therapeutic effect.

The agents of this invention may also be administered orally to the patient, in a manner such that the concentration of drug is sufficient to achieve one or more of the therapeutic indications disclosed herein. Typically, a pharmaceutical composition containing the agent is administered at an oral dose of between about 0.1 to about 50 mg/kg in a manner consistent with the condition of the patient. Preferably the oral dose would be about 0.5 to about 20 mg/kg.

The agents of this invention may be tested in one of several biological assays to determine the concentration of an agent, which is required to have a given pharmacological effect.

### Kit of Parts

Another aspect of the invention relates to a kit comprising an Slfn11 inhibitor, anti-Slfn11 antibody, nucleic acid probe for Slfn11 or at least one QPCR primer for Slfn11, for use in any of the above-described methods.

### Diagnostics and Prognostics

The invention also relates to the use of Slfn11 as a biomarker in the diagnosis or prognosis of diseases characterized by proliferative activity, particularly in individuals being treated with Axl or Akt3 inhibitors.

As used herein, the term “prognostic method” means a method that enables a prediction regarding the progression of a disease of a human or animal diagnosed with the disease, in particular, cancer. More specifically, the cancers of interest include acute myelocytic leukemias (AMLs), breast, lung, gastric, head and neck, colorectal, renal, pancreatic, uterine, hepatic, bladder, endometrial and prostate cancers and other leukemias.

The term “diagnostic method” as used herein means a method that enables a determination of the presence or type of cancer in or on a human or animal. Suitably the marker allows the success of treatment with an Axl, Akt3, or Slfn11 inhibitor to be assessed. As discussed above, suitable diagnostics include probes directed to any of the genes as identified herein such as, for example, QPCR primers, FISH probes and so forth.

The term “prognostic method” as used herein means a method that enables a determination of the likelihood of a subject being susceptible or responsive to treatment with a particular agent/regimen. Such prognostic methods provide information on the likely outcome of a particular treatment regimen, for example, the likelihood of a subject responding to said treatment, and/or information as to how aggressively an individual should be treated within a particular treatment regimen, and/or how aggressively an individual should be treated with conventional therapeutic methods such as radiation/chemotherapy. The prognostic methods described herein therefore have important applications in the field of personalized medicines.

One preferred embodiment thus relates to the use of a biomarker as described above in a personalized medicine application.

In one preferred embodiment, the personalized medicine application is for determining whether a subject will be susceptible or responsive to treatment with an Akt3 or Axl inhibitor.

In one preferred embodiment, the personalized medicine application is for determining whether a subject is particularly likely to suffer from metastatic cancer. In some embodiment a high level of Slfn11 expression in tumour cells as compared to non-tumour cells is indicative of an increased risk of suffering from metastatic cancer. In some embodiments the expression of Slfn11 is considered to be “high” when the level of expression in non-tumour cells is no more than 90% of the level in tumour cells, such as no more than 80%, no more than 70%, no more than 60%, no more than 50%, no more than 40%, no more than 30%, no more than 20%, or no more than 10% of the level of the level in tumour cells.

Another aspect of the invention relates to a prognostic method for determining whether a subject will be susceptible to treatment with an Akt3, Axl, or Slfn11 inhibitor, said method comprising detecting the occurrence of epithelial-to-mesenchymal transition (EMT) in said subject.

Another aspect of the invention relates to the use of Slfn11 as a biomarker in a prognostic agent for determining whether a subject will be susceptible or responsive to treatment with an Akt3, Axl, or Slfn11 inhibitor. For example, in some embodiments, an increased level of Slfn11 in a tumour sample relative to a non-tumour control indicates the subject is susceptible or responsive to treatment with an Akt3, Axl, or Slfn11 inhibitor.

For example, in some embodiments the level of Slfn11 expression in a subject is assessed following treatment of the subject with an Axl or Akt3 inhibitor (such as BGB324) wherein subjects having reduced expression of Slfn11 following treatment are considered to be susceptible or responsive to treatment.

In some embodiments the treatment is a single dose of Axl or Akt3 inhibitor (such as BGB324) at 2 mg/kg, with samples analysed 24 hours after treatment. In some embodiments the treatment is a single dose of Axl or Akt3 inhibitor (such as BGB324) at 5 mg/kg, with samples analysed 24 hours after treatment. In some embodiments the treatment is a single dose of Axl or Akt3 inhibitor (such as BGB324) at 10 mg/kg, with samples analysed 24 hours after treatment. In some embodiments the treatment is a single dose of Axl or Akt3 inhibitor (such as BGB324) at 20 mg/kg, with samples analysed 24 hours after treatment. In some embodiments the treatment is a single dose of Axl or Akt3 inhibitor (such as BGB324) at 50 mg/kg, with samples analysed 24 hours after treatment. In some embodiments the treatment is a single dose of Axl or Akt3 inhibitor (such as BGB324) at 100 mg/kg, with samples analysed 24 hours after treatment.

In some embodiments the treatment is a single daily dose of Axl or Akt3 inhibitor (such as BGB324) at 2 mg/kg for 14 days, with samples analysed 24 hours after the final treatment. In some embodiments the treatment is a single daily dose of Axl or Akt3 inhibitor (such as BGB324) at 5 mg/kg for 14 days, with samples analysed 24 hours after the final treatment. In some embodiments the treatment is a single daily dose of Axl or Akt3 inhibitor (such as BGB324) at 10 mg/kg for 14 days, with samples analysed 24 hours after the final treatment. In some embodiments the treatment is a single daily dose of Axl or Akt3 inhibitor (such as BGB324) at 20 mg/kg for 14 days, with samples analysed 24 hours after the final treatment. In some embodiments the treatment is a single daily dose of Axl or Akt3 inhibitor (such as BGB324) at 50 mg/kg for 14 days, with samples analysed 24 hours after the final treatment. In some embodiments the treatment is a single daily dose of Axl or Akt3 inhibitor (such as BGB324) at 100 mg/kg for 14 days, with samples analysed 24 hours after the final treatment.

In some embodiments the expression of Slfn11 is considered to be “reduced” when the level of expression after treatment is no more than 90% of the level before treatment, such as no more than 80%, no more than 70%, no more than 60%, no more than 50%, no more than 40%, no more than 30%, no more than 20%, or no more than 10% of the level before treatment.

A related aspect of the invention relates to the use of Slfn11 as a biomarker in a prognostic agent for determining whether a subject will be susceptible or responsive to treatment with an Akt3, Axl, or Slfn11 inhibitor. For example, in some embodiments the level of Slfn11 expression in a subject is assessed following treatment of the subject with an Axl or Akt3 inhibitor (such as BGB324) wherein subjects having increased expression of Slfn11 following treatment are considered to be resistant or unresponsive to treatment.

In some embodiments the treatment is a single daily dose of Axl or Akt3 inhibitor (such as BGB324) at 2 mg/kg for 14 days, with samples analysed 72 hours after the final treatment. In some embodiments the treatment is a single daily dose of Axl or Akt3 inhibitor (such as BGB324) at 5 mg/kg for 14 days, with samples analysed 72 hours after the final treatment. In some embodiments the treatment is a single daily dose of Axl or Akt3 inhibitor (such as BGB324) at 10 mg/kg for 14 days, with samples analysed 72 hours after the final treatment. In some embodiments the treatment is a single daily dose of Axl or Akt3 inhibitor (such as BGB324) at 20 mg/kg for 14 days, with samples analysed 72 hours after the final treatment. In some embodiments the treatment is a single daily dose of Axl or Akt3 inhibitor (such as BGB324) at 50 mg/kg for 14 days, with samples analysed 72 hours after the final treatment. In some embodiments the treatment is a single daily dose of Axl or Akt3 inhibitor (such as BGB324) at 100 mg/kg for 14 days, with samples analysed 72 hours after the final treatment.

In some embodiments the expression of Slfn11 is considered to be “increased” when the level of expression before treatment is no more than 90% of the level after treatment, such as no more than 80%, no more than 70%, no more than 60%, no more than 50%, no more than 40%, no more than 30%, no more than 20%, or no more than 10% of the level after treatment.

Another aspect of the invention relates to a prognostic method for determining whether a subject is particularly likely to suffer from metastatic cancer, said method comprising detecting the occurrence of epithelial-to-mesenchymal transition (EMT) in said subject.

In some embodiments, the occurrence of epithelial-to-mesenchymal transition (EMT) in said subject is determined by assessing the level of Slfn11 activity or expression, wherein Slfn11 activity or expression, or increased Slfn11 activity or expression, is indicative of the occurrence of epithelial-to-mesenchymal transition (EMT).

In some embodiments the expression of Slfn11 is considered to be “increased” when the level of expression in non-tumour cells is no more than 90% of the level in tumour cells, such as no more than 80%, no more than 70%, no more than 60%, no more than 50%, no more than 40%, no more than 30%, no more than 20%, or no more than 10% of the level of the level in tumour cells.

Throughout the specification, preferably the methods described herein are performed in vitro or ex vivo.

Throughout the specification, references are made to Slfn11 inhibitors, Slfn11 activators, and Slfn11 modulators. As used herein, the term Slfn11 inhibitor refers to an agent that inhibits or reduces Slfn11 activity or expression; the term Slfn11 activator refers to an agent that activates or increases Slfn11 activity or expression; and the term Slfn11 modulator encompasses both Slfn11 inhibitors and Slfn11 activators.

The invention will now be described in more detail, by way of example and not limitation, by reference to the accompanying drawings. Many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

### BRIEF DESCRIPTION OF THE DRAWINGS

FIG. 1 shows a single gene graph describing regulation of SLFN11 in response to 14d BGB324-treatment in mice bearing subcutaneous MV4-11 xenografts. The transcript is 3.98-fold decreased from control-level in the tumors from mice treated with the highest dose (50 mg/kg);

FIG. 2 shows a comparison of the expression levels of Axl and SLFN11. The comparison demonstrates that these two proteins co-localize in normal blood myeloid cells (red circle);

FIG. 3 shows Slfn11 expression in PBMCs. The mouse anti-human Slfn11 antibody was diluted 1:50-1:600 and used for staining of fixed PBMCs. Figure shows representative histograms of stained PBMCs compared to secondary antibody/unstained control. Geometric mean is calculated based on fluorescent measurements of 50 000 cells. Dotplots show Slfn11 positive cells in 3 different blood subpopulations;

FIG. 4 shows Slfn11 and Axl coexpression in healthy blood. A. Flow cytometry scatter plot of Axl (FL-4 ch) and Slfn11 (F1-1 ch) stained PBMCs from healthy volunteers. Left panel (A01)) is unstained sample and the Q1-4 gate has been set according to this sample. Middle panel (A05) is secondary ab control which function as a control to verify that all negative cells are located in quadrant 4 (Q4). Right panel (A03) is Axl/Slfn11 costained sample showing Axl+ cells (Q1), Axl+/Slfn11+ cells (Q2), Slfn11+ cells (Q3) and negative cells (Q4). B. Backgating of samples showing where the stained samples are localized in the viable PBMC population. Left panel is Axl+ cells, middle panel is Axl+/Slfn11+ cells whereas right panel is Slfn11+ cells only;

FIG. 5 shows Slfn11 expression in AML cell lines. Western blot analysis (A) showing Slfn11 expression levels in inhouse AML cell lines MOLM-13, OCI-M1, OCI-AML3 and Kasumi cells. HeLa cell lysate serve as a negative control.

The Slfn11 expression levels in these cells were confirmed by flow cytometry (B) using the same Slfn11 primary antibody and AlexaFluor488 conjugated secondary antibody. Data is representative for 2 experiments (10 000 cells per measurement);

FIG. 6 shows a western blot of MV4-11 xenografts from mice treated for 14 days with 25 or 50 mg/kg, or with a single high dose (100 mg/kg) of BGB324.

FIG. 7 shows a western blot of MV4-11 xenografts from mice treated with a single low (50 mg/kg, upper panels) or high (100 mg/kg, lower panels) dose of BGB324. The tumors were harvested at different time points after treatment, from 4-up to 72 hours.

FIG. 8 shows the reduction of SLFN11 expression levels after BGB324 treatment. AML cell lines were treated with BGB324 (IC50 values) for 24 h, 48 h and 72 h and analysed by flow cytometry. A representative flow histograms for MOLM-13, OCI-AML3 and OCI-M1 after treatment for 24 h (upper panels), 48 h (mid panels) and 72 h (lower panels) showing control cells (red), BGB324 treated cells (blue), secondary antibody control (green) and unstained sample (pink). B. Flow cytometric analysis of Slfn11 expression after BGB324 treatment of Kasumi (n=1), OCI-M1 (n=2), OCI-AML3 (n=2), OCI-AML5 (n=1) and MOLM-13 wt (n=3), MOLM-13shLuc (n=1) and MOLM-13shAxl (n=2). Data show geometric mean (% of control+−S.D. of 2-3 experiments.

FIG. 9 shows the results of treatment with BGB324 show a dose-dependent downregulation of Slfn11 in MOLM-13 wt cells. MOLM-13 cells were treated with BGB324 for 48 hours at concentrations ranging from 0.1 uM to 1.2 uM. Slfn11 expression levels were analysed by western blot (A) and flow cytometry (B) using mouse anti-human Slfn11 antibody.

FIG. 10 shows the response of total SLFN11 levels in AML cell lines after 24, 48 and 72 hours of treatment with BGB324 at 0.05, 0.1 or 0.3 μM (MOLM13 and Mv4-11, upper panels) or at 2.5 μM (Kasumi and OCI-AML5, lower panels). The graphs show geometric mean of fluorescence, calculated as % of control (which is set to 100%—indicated by a dotted line), ±SEM. * indicates significance relative to control, calculated using a two-tailed Student's t-test. * p<0.05, **p<0.005, n=3.

FIG. 11 shows spleen, bone marrow and blood from mice stained with anti-human-CD33 and -CD45 antibodies to identify leukemic cells in the tissues. CD33/CD45 double-positive cells were quantified as % of total live cell count (A). Bone marrows and spleens from treated and non-treated mice were assessed for biomarker expression by flow cytometry. The samples were stained with anti-human CD33 antibody, and biomarker expression was only evaluated in CD33-positive cells (B). The graphs show geometric mean of fluorescence, calculated as % of control (which is set to 100%), ±SEM. * indicates significance relative to control, calculated using a two-tailed Student's t-test. * p<0.05, **p<0.005, n>5.

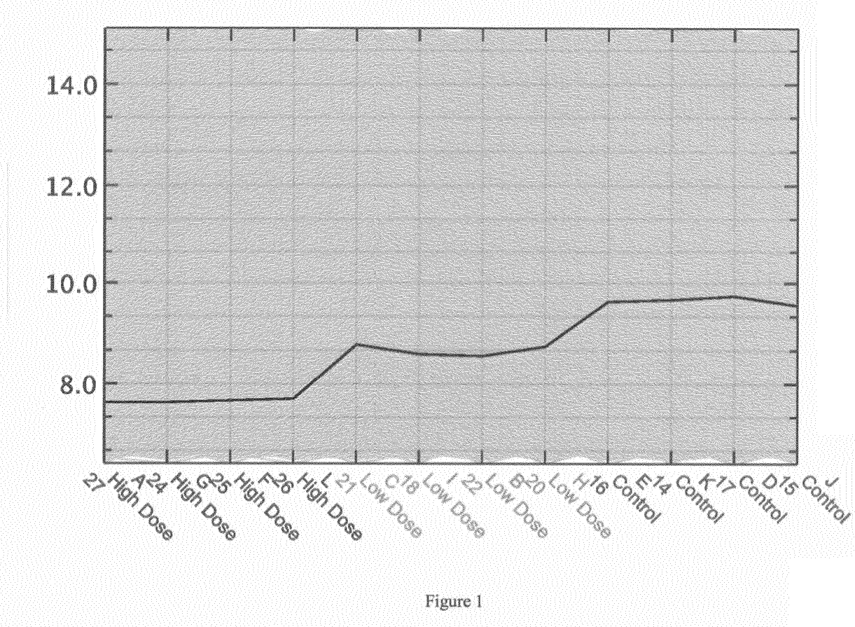

### MATERIAL AND METHODS

### Materials

10% NuPAGE® Bis-Tris precast gels (#NP0301BOX, Invitrogen)
Amersham Hybond-P PVDF transfer membrane (#RPN303F, GE Healthcare)
BGB324 (Manufacturer: Almac Group, N Ireland. Lot #011-SR-324 DA2aI-15, 10 mM in DMSO, prepared 17.02.14 by Tone Sandal)
BD Phosflow™ Lyse/Fix Buffer 5× (#558049, BD Biosciences)
Complete Mini Protease Inhibitor Cocktail tablets (#04693116001, Roche)
ECL-reagents: Reagent1 and Reagent2 (#1859701 and #1859698, Thermo Scientific)
Fetal Bovine Serum (FBS, #A9647, Sigma)
MagicMark™ XP Western Protein Standard (#LC5602, Invitrogen)
Mouse-anti-human Axl Ab (1H12-1B7-5D6, BerGenBio, BGB #47)
Mouse-anti-human Axl Ab (1H12-1B7-5D6, BerGenBio, BGB #47) Alexa 647-conjugated (1.2 mg/ml in PBS. Stock: 22 Oct. 2013, made by Hallvard Haugen)
Mouse-anti-human SLFN11 AB (sc-374339, Santa Cruz, BGB #91)
Nitrocellulose membrane, Whatman Protran BA85 (#10401196, GE Healthcare)
NP-40: Pierce IP lysis buffer (#87788, Thermo Scientific)
NuPAGE Antioxidant (#NP0005, Invitrogen)
NuPAGE LDS Sample Buffer 4× (#NP0007, Invitrogen)
phosSTOP Phosphatase Inhibitor Cocktail tablets (#04693116001, Roche)
Pierce BCA protein assay kit (#PI-23227, Thermo Scientific)
Rabbit anti actin Ab (#A5060, Sigma-Aldrich)
Rabbit anti-human GAPDH [EPR6256] AB (#ab128915, Abcam)
SeeBlue® Plus 2 Pre-Stained Standard (#LC5625, Invitrogen)
AlexaFluor 488 goat anti-mouse IgG (H+L) (#A11029, Invitrogen)
Goat anti-mouse IgG (H+L), Horseradish peroxidase conjugate (#G-21040, Life Technologies)

### Cells

MOLM13 cells were grown in RPMI-1640 media (R8758, Sigma-Aldrich), supplemented with 10% fetal bovine serum (FBS), L-glutamine (4 mM) and penicillin-streptomycin (5 μg/ml).

Mv4-1/cells (ATCC, CRL9591) were grown in Isovec s Modified Dulbecco's Medium (IMDM; #30-2005, ATCC) supplemented with 10% fetal bovine serum (FBS), L-glutamine (4 mM), streptomycin (5 μg/ml) and penicillin (5 U/ml).

OCI-M1 cells were grown in were grown in Isovec's Modified Dulbecco's Medium (IMDM: #30-2005, ATCC) supplemented with 5% fetal bovine serum (FBS), L-glutamine (4 mM), streptomycin (5 μg/ml) and penicillin (5 U/ml).

OCI-AML3 cells were grown in Alpha MEM (#22561-021, Gibco by Life Technologies) supplemented with 20% fetal bovine serum (FBS L-glutamine (4 mM), streptomycin (5 μg/ml) and penicillin (5 U/ml).

OCI-AML5 cells were grown in Alpha MEM (#22561-021, Gibco by Life Technologies) supplemented with 20% fetal bovine serum (FBS), L-glutamine (4 mM), streptomycin (5 μg/ml), penicillin (5 U/ml), and GM-CSF (2.5 ng/ml).

Kasumi cells were grown in RPMI-1640 media (R8758, Sigma-Aldrich) supplemented with 20% fetal bovine serum (FBS), L-glutamine (4 mM), streptomycin (5 μg/ml) and penicillin (5 U/ml).

### Methods

### Western Blot, General Protocol

For western blot analysis, cells were lysed on ice using NP-40 lysis buffer with protease- and phosphatase inhibitors. Total protein concentration in lysates was measured using a BCA protein assay kit following the manufacturers instructions. 10% NuPAGE® Bis-Tris precast gels were loaded with 30-50 μg of protein in each well diluted in sample buffer and antioxidant. A 1:1 mix of MagicMark™ XP Western Protein Standard (Invitrogen) and SeeBlue® Plus 2 Pre-Stained Standard was used as protein standard. Gels were run at 50V for 20 min, then at 100V for 1 h30 min. Blotting was done on ice for 1 h 30 min at 100V onto PVDF (pre-activated with MeOH) or nitrocellulose membranes. Membranes were washed in TBS-0.1% Tween-20 (TBS-T), and blocked in TBS-T 5% BSA for at least 1 h at RT. Primary antibody was added at 1:1000 in TBS-T 5% BSA (rabbit-anti-actin Ab was added at 1:2000, rabbit anti-GAPDH Ab was added at 1:3000) in TBS-T 5% BSA, and membranes were incubated over night at 4° C. Membranes were then washed 3× in TBS-T, and incubated in HRP-conjugated secondary antibody at 1:5000 dilution in TBS-T 5% milk for 45 min at RT. Membranes were developed for 1 min using ECL-reagents and imaged with chemiluminescence using a Molecular Imager ChemiDoc™ XRS (BioRad).

All incubation- and washing steps were done on a roller.

### Staining of Cells for Flow Cytometry, General Protocol

Live cells were centrifuged at 300 g for 5 minutes, washed once with PBS and centrifuged again. Cells were then fixed in 4% PFA in PBS for 10 minutes at 37° C., and resuspended in PBS. Unless processed immediately, samples were at this point stored in PBS at 4° C. (up to three weeks) or at −80° C. (for long-term storage). If intracellular epitopes were stained, cells were permebealized in 90% MeOH for 30 minutes on ice. Unless processed immediately, samples were also be stored in 90% MeOH at −20° C. for up to two months.

### Further Staining Procedure (Permebealized or Non-Permebealized Cells)

Cells (150-200 000 per sample) were washed 1× in PBS and incubated in blocking buffer; (PBS+0.5% BSA) for 15 minutes at room temperature. Thereafter, cells were incubated with primary mouse-anti-human SLFN11 Ab at the indicated dilutions in incubation buffer (IB, PBS+0.5% BSA) for 1 hour at room temperature. Cells were then washed 3× with IB and incubated with secondary AB (conjugated to a fluorescent flurophore) at 1:1000 dilution in IB for 30 minutes at room temperature. Finally, cells were washed 3× in IB and resuspended in PBS. For costaining experiments, cells were subsequently incubated with mouse-anti-human Axl Ab (1H12-A647 conjugated Ab) diluted 1:3000 in IB. Cells were analyzed immediately, or stored for up to 24 hours at 4° C. before analysis.

Analysis of cells was done on a BD LSR Fortessa or a BD C6 Accuri flow cytometer, and further processing was done using FlowJo v.7.6.

All incubation steps were done on a spinning wheel or gentle shaker.

### Staining of Blood for Flow Cytometry, General Protocol

Human blood from healthy donors was collected in the presence of sodium citrate and mixed with 20 volumes of pre-warmed BD phosflow lyse/fix buffer (diluted to 1× in distilled water), followed by incubation in 37° C. water bath for 10 minutes. Cells were spun at 500 g for 8 minutes and washed once with PBS. Cells were permeabilized by adding 70% MeOH followed by 30 min incubation on ice. Unless processed immediately, samples were at this point stored in MeOH at −20° C. for up to 4 weeks.

Prior to antibody staining, cells were spun at 600 g, washed twice and resuspended in IB buffer. Cells were aliquoted into volumes corresponding to 100 ul collected blood (before dilution), and stained as described above (section two in “Staining of cells for flow cytometry, general protocol”).

All washing and incubation steps were done on a spinning wheel or gentle shaker.

### EXAMPLES

### Example 1: SLFN11 is Reduced in MV4-11 Xenografts after BGB324 Treatment

To identify new potential biomarkers of BGB324 efficacy in AML, microarray analyses of tumor material from several AML studies were performed. In the first two studies, NOD/SCID mice were implanted with subcutaneous Mv4-11 xenografts. In study 1 the mice were treated with control (vehicle) or a single dose of BGB324 (50 or 100 mg/kg), and sacrificed after 24 hours. In study 2 the mice were treated BID for 14 days with control (vehicle), low (25 mg/kg) or high (50 mg/kg) dose of BGB324 for 14 days. In a third study, MOLM13 cells were injected intravenously into the tail vein of NSG mice. After three days of inoculation, the animals were treated with control (vehicle), high (50 mg/kg) or low (25 mg/kg) dose of BGB324 QD until the vehicle-treated animals were moribund (14 days after initiation of treatment). Development of leukemia was confirmed by flow cytometric analyses of human CD33/CD45-positive cells in the blood, spleens and bone marrow, and RNA isolated from spleens were sent for microarray analysis.

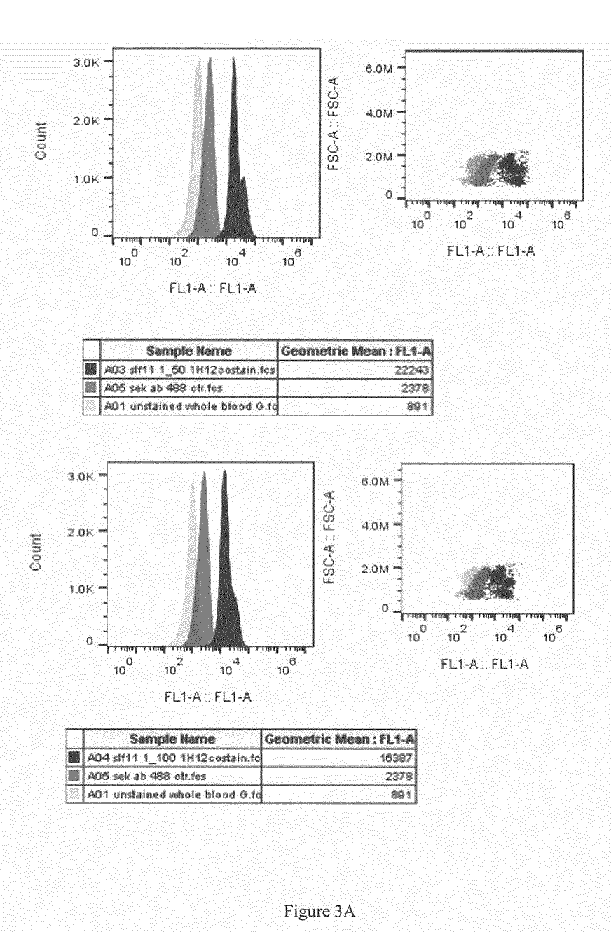

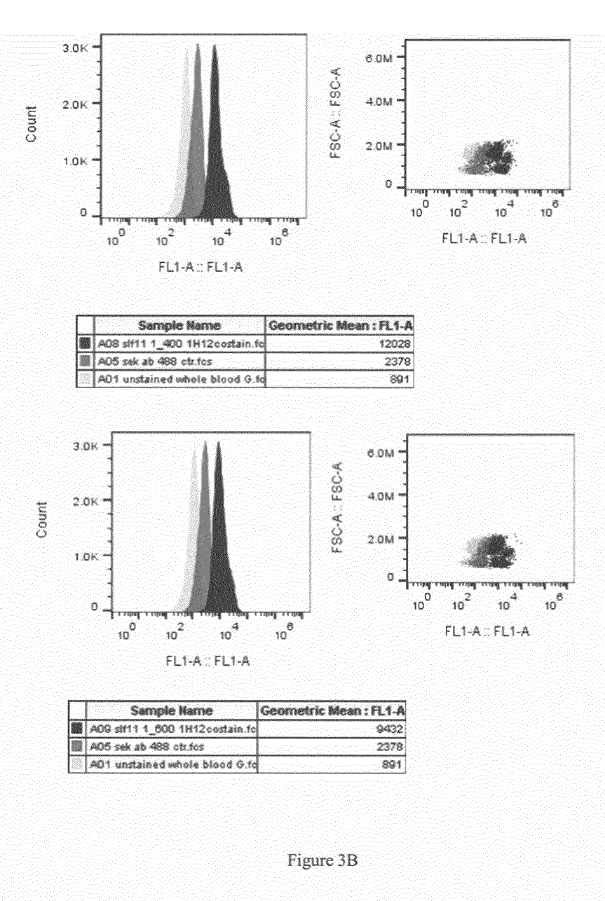

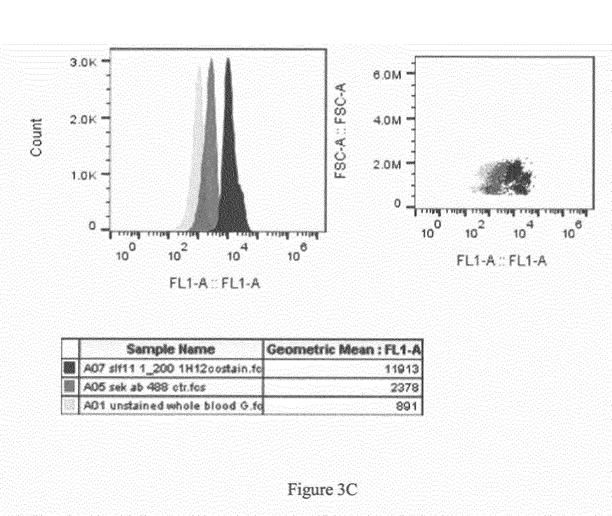

One of the most interesting hits from a Rank Product Analysis of study 2 was a transcript called SLFN11. In this study, a highly significant dose-dependent downregulation of SLFN11 after 14d treatment was found (25 mg/kg: 1.98-fold downregulation, q=0, rank #3, 50 mg/kg: 3.98-fold downregulation, q=0, rank #9) (FIG. 3). A Rank Product Analysis of study 1 (single dose treatment) also revealed a significant reduction of SLFN11, although it was not one of the top hits in this dataset (1.449-fold, q=0, rank #245).

Furthermore, a SAM (significance of microarray) analysis of the same experiment (study 2) showed reduction of SLFN11 as the most significant (#1) hit (q=0.0) after BGB324 treatment. However, in the systemic AML study (study 3), SLFN11 was not among the transcripts that were significantly downregulated after BGB324-treatment.

### Example 2: AXL and SLFN11 are Co-Expressed in Blood Myeloid Cells

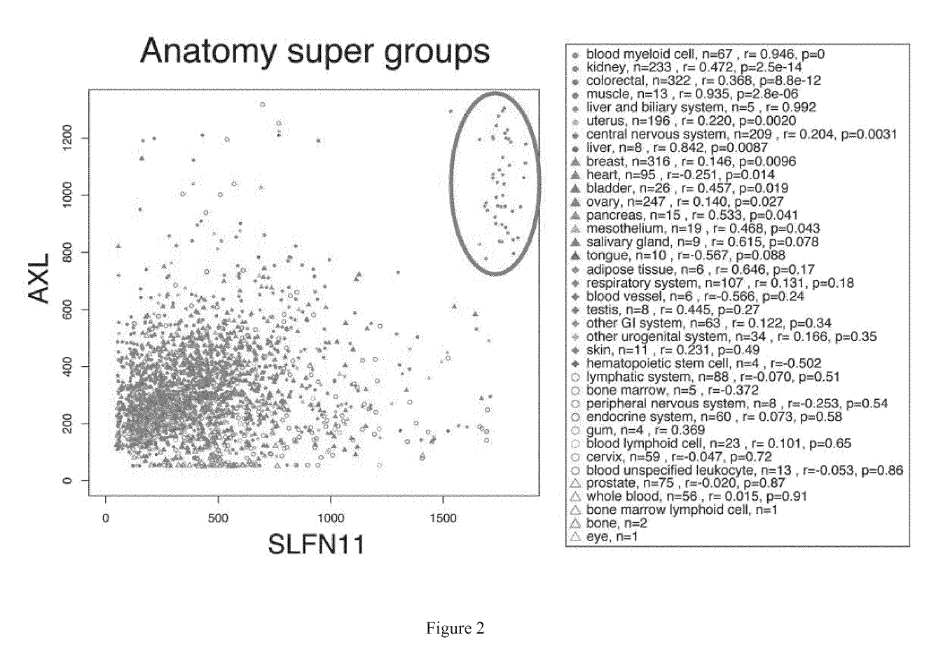

The results from the microarray analyses strongly suggest that reduction of the SLFN11 transcripts in MV4-11 tumors is a direct consequence of Axl inhibition by BGB324. Thus, there might be crosstalk between these two proteins. However, there are currently no publications linking Axl and Slfn11 functionally. Therefore, it was decided to investigate the expression levels of these two proteins in different tissues using the GeneSapiens website (http://ist.medisapiens.com/, IST4 database). GeneSapiens offers a freely available fully integrated and annotated online database of the human transcriptome, where the expression levels of genes can be compared in healthy tissue as well as in tumor tissue and cell lines. A comparison of AXL and SFLN11 gene expression showed that AXL and SLFN11 has a high level of co-expression in normal blood myeloid cells (FIG. 2), suggesting that Axl and Slfn11 might also have a functional relationship in these cells.

### Example 3: Slfn11 is Expressed in Various PBMC Subpopulations

To evaluate the result from the database comparison, the expression of Slfn11 and Axl was investigated in blood samples from healthy donors. Blood was collected from a healthy volunteer (id:G) in sodium citrate vacutainer. The blood was fixed and PBMCs were prepared for flow. As an initial experiment, Slfn11 mouse monoclonal antibody was tested on blood by performing a antibody dilution series in the range from 1:50-1:600. By flow cytometric analysis it was found that Slfn11 expression in various subpopulations of blood (FIG. 3), and dilution 1:100 was chosen as standard dilution for further experiments staining PBMCs.

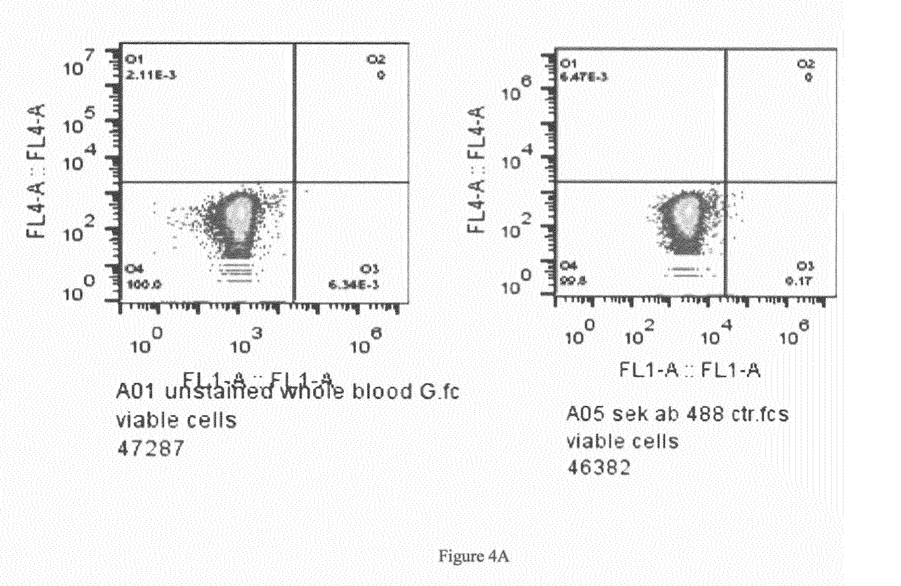

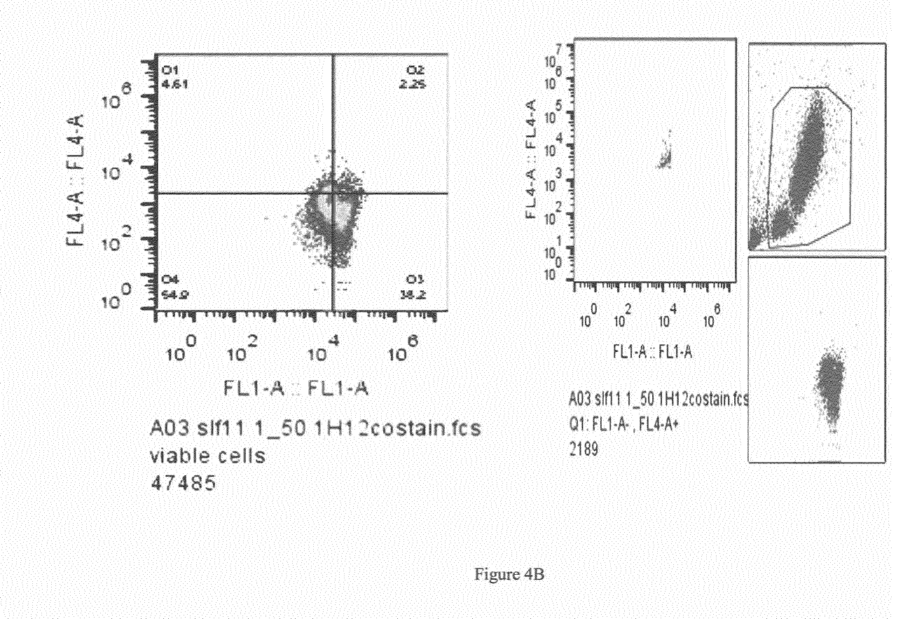

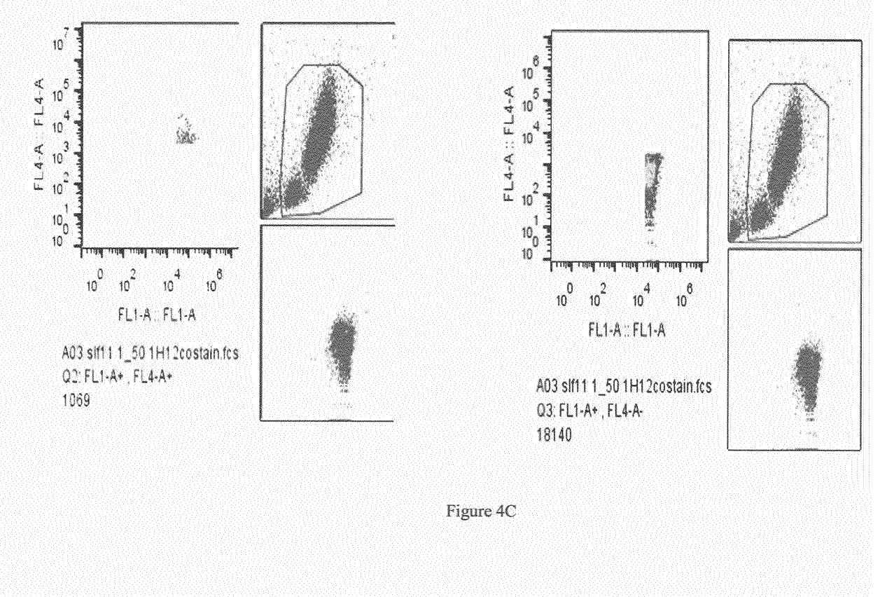

Next, it was investigated if Slfn11 and Axl were coexpressed in any of the different blood subpopulations. PBMCs were prepared as above, stained with Slfn11 Ab (1:100) and secondary antibody Alexa488 (1:1000), followed by staining with 1H12-Alexa 647 conjugated antibody (1:3000). By flow cytometric analysis expression of both Axl and Slfn11 in PBMCs was found. However, coexpression of Axl and Slfn11 was only identified in a small subpopulation of cells (FIG. 4).

As seen in FIG. 4, it should be noted that coexpression of Slfn11 and Axl is mostly seen in the population which appears to be granulocytes, although the identity of these cells needs to be confirmed with immunocell-specific CD-marker staining (see discussion for further explanation).

### Example 4: Slfn11 Expression in AML Cells Lines

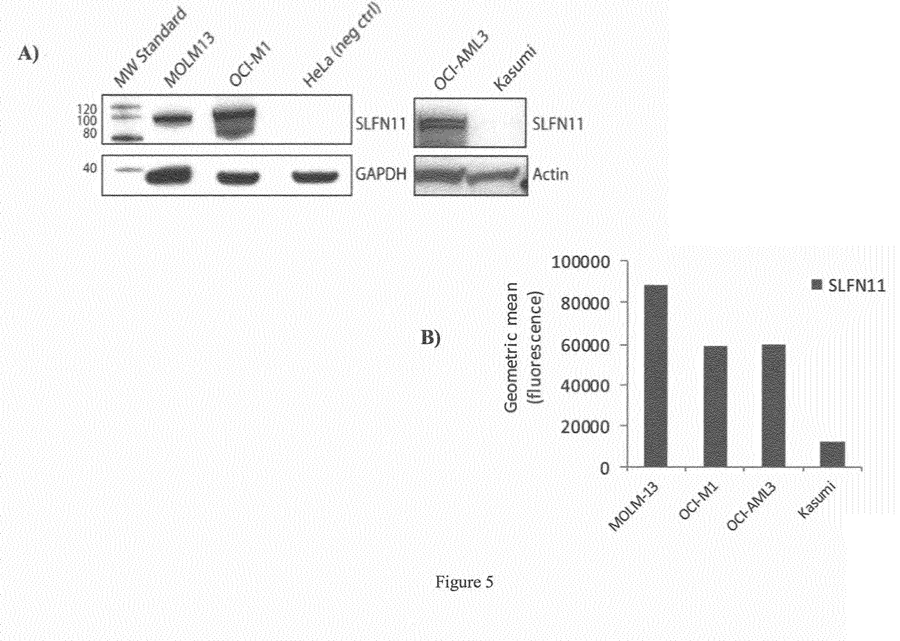

After confirming the expression of Slfn11 in normal blood PBMCs, it was decided to further investigate the expression level of Slfn11 in AML cells. A panel of AML cell lines were examined both by flow cytometry and western blot. Western blot revealed Slfn11 expression in several of the AML lines, including MOLM13, OCI-M1 and OCI-AML3, but not in Kasumi cells (FIG. 5A). HeLa cells were included as a negative control, as these cells have been shown not to express SLFN11.

These results were subsequently confirmed by flow cytometry using the same antibody. As can be observed in FIG. 5B, flow cytometric analysis shows a low, but detectable level of Slfn11 in Kasumi cells, although this is not detected by Western Blotting (FIG. 5A).

### Example 5: Slfn11 Expression is Reduced in MV4-11 Tumor Xenografts after BGB324 Treatment

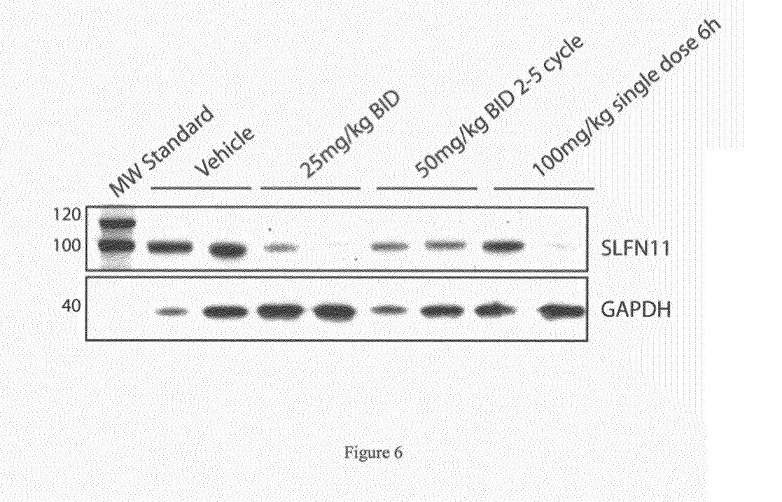

The data from microarray analyses of MV4-11 tumor xenografts showed a significant reduction of the SLFN11 transcripts in the tumor cells after both long-term treatment as well as treatment with only a single dose of BGB324. To confirm whether this also resulted in a subsequent reduction of the Slfn11 protein in these tumors, lysates of MV4-11 subqutaneous tumor xenografts from both single dose and continuous treatment were examined by western blot. In these studies, mice had been treated with BGB324 25 mg/kg BID and 50 mg/kg BID 2-5 cycle for 14 days, or with a single dose 100 mg/kg (tumors harvested 6 h after treatment). In these tumors, a marked reduction of Slfn11 compared to control levels was found after 14 days of treatment. In tumors from mice treated with only a single high (100 mg/kg) dose of BGB324, Slfn11 was reduced in one of the two parallel samples examined (FIG. 6).

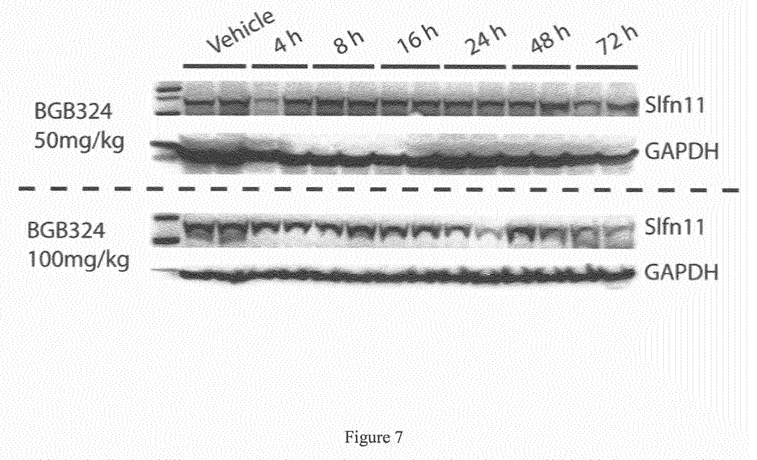

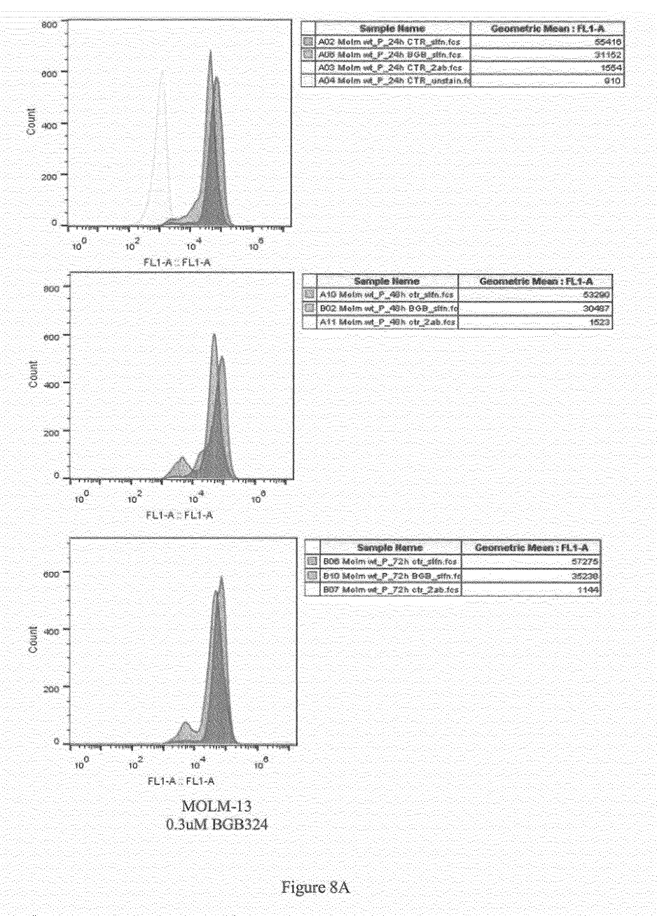

Since Slfn11 was reduced in only one of two parallel tumor samples after a single treatment, it was decided to examine more lysates from Study 1 by western blot. In this study, mice with subcutaneous MV4-11 tumor xenografts were treated with a single low (50 mg/kg) or high (100 mg/kg) dose of BGB324, and tumors were harvested at different time points, from 4 hours up to 72 hours after treatment. A slight reduction of Slfn11 could be detected 72 hours after treatment in mice treated with a low dose (50 mg/kg) of BGB324 (FIG. 7, upper panels). In mice treated with a high dose (100 mg/kg) of BGB324, reduction of Slfn11 in tumors 24, 48 and 72 hours after treatment was observed (FIG. 7, lower panels), but not at earlier time points (unlike the results from FIG. 6). Thus, it appears that Slfn11 is reduced in MV4-11 xenografts within 24-72 hours (depending on the drug concentration) after treatment with a single dose of BGB324. The effect of BGB324 on Slfn11 expression in these tumors appears to be long-lasting, as the protein level stays low 72 hours after treatment. However, there appear to be individual differences, and more than two parallel samples of each time point need to be examined in order to better understand the dynamics of Slfn11 reduction in MV4-11 xenografts after BGB324-treatment.

### Example 6: Slfn11 Expression is Reduced in AML Cell Lines after BGB324 Treatment

Microarray and western blot results from MV4-11 xenografts show downregulation of SLFN11 after BGB324 treatment. This led to the investigation of whether other AML cell lines also exhibited Slfn11 reduction after BGB-324 treatment in vitro.

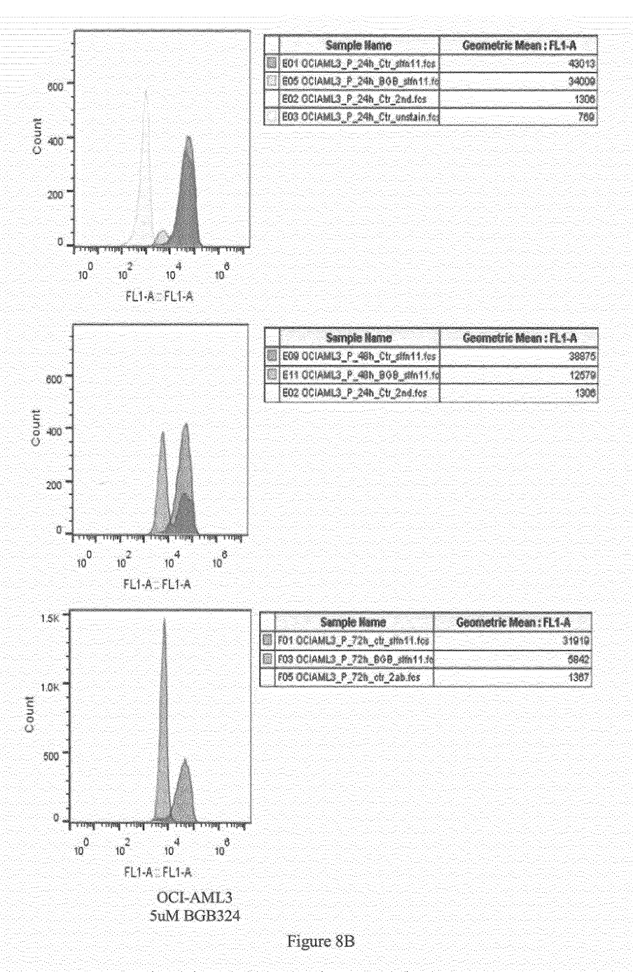

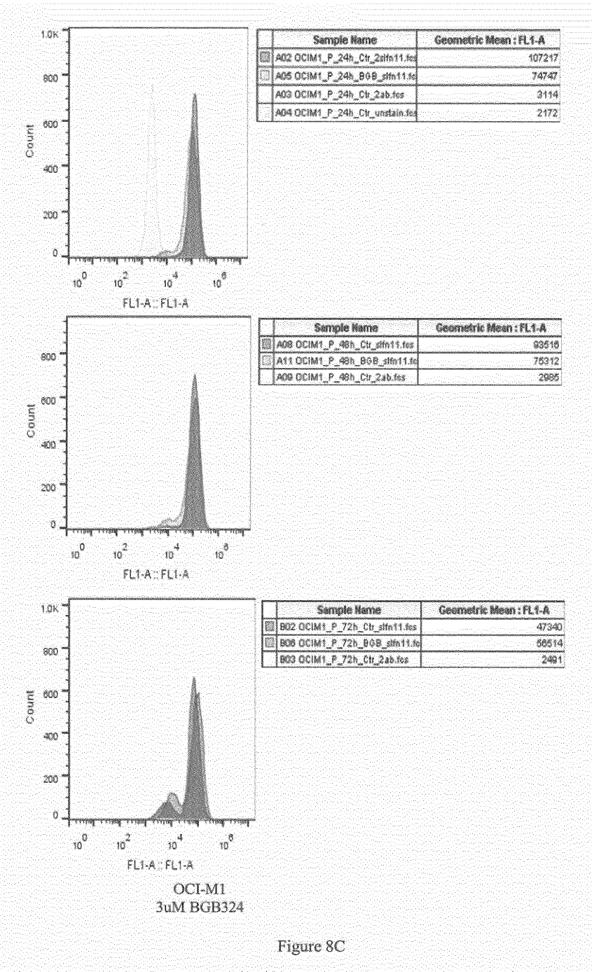

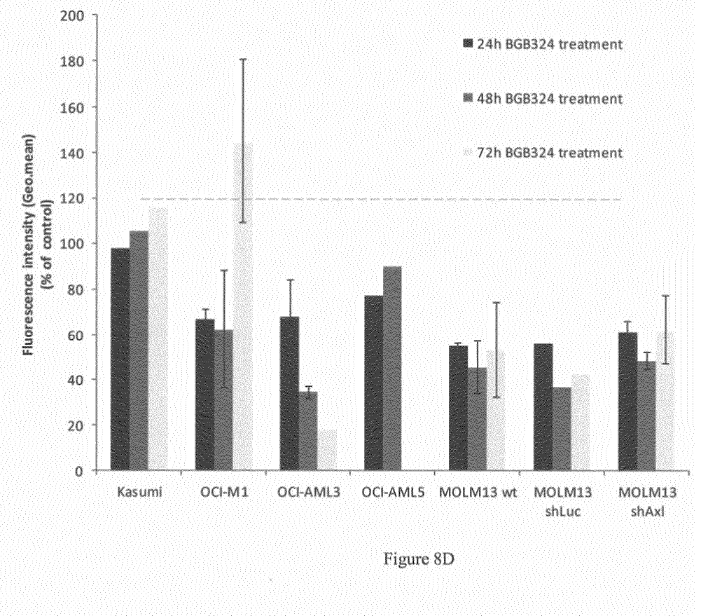

MOLM-13, OCI-M1 and OCI-AML3 cells were seeded at different cell densities (corresponding to length of treatment) and treated with BGB324 at IC50-values for 24, 48 and 72 hours. By flow cytometry analysis it was shown that treatment with BGB24 resulted in a marked reduction of SLFN11 expression at nearly all time points in these cells (FIG. 8).

Next, the dose-dependence of the reduction in Slfn11 expression on BGB324 treatment was investigated.

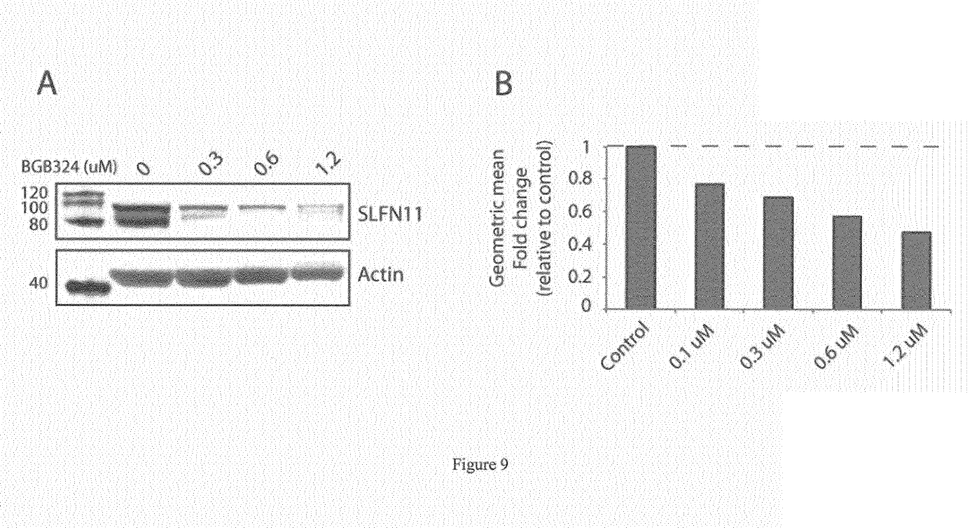

MOLM-13 cells treated with increasing doses of BGB324 for 48 hours showed a dose-dependent reduction in Slfn11 expression by western blotting (FIG. 8A). These results were confirmed by flow cytometry (FIG. 9B) using the same antibody.

### Example 7: Total Protein Expression of SLFN11 in the AML Cell Panel after Long-Term Treatment (24, 48 and 72 Hours) with BGB324

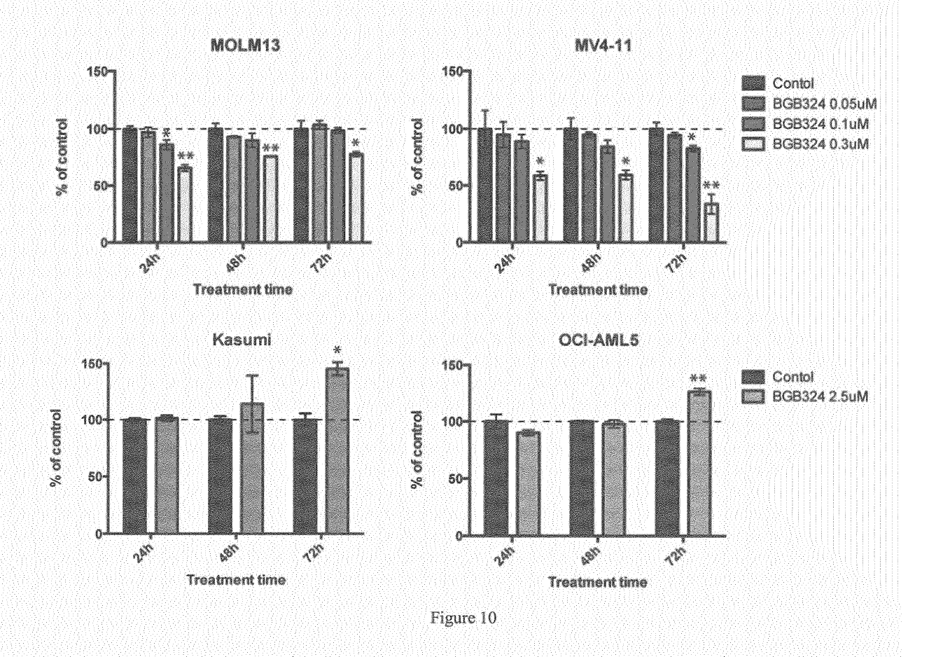

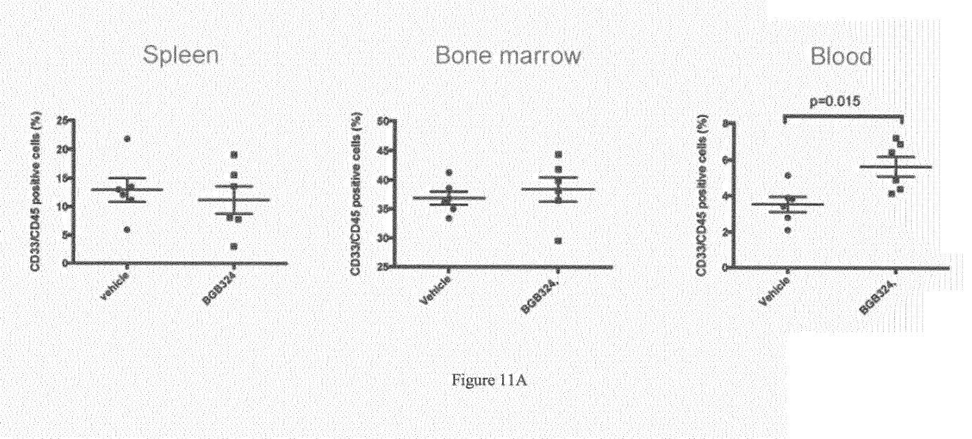

When examining total protein expression of SLFN11 in the AML cell panel after long-term treatment (24, 48 and 72 hours) with BGB324, a significant reduction was found at all time points in MOLM13 and Mv4-11 at the highest treatment dose (0.3 μM) (FIG. 10).

In Mv4-11, SLFN11 was also significantly reduced at treatment with 0.1 uM BGB324 for 72 hours. In Kasumi and OCI-AMLS, the opposite was found; a significant increase of SLFN11 expression, but only after 72 hours of treatment. At 24 and 48 hours, there was no significant differences between treated and control cells.

Thus, SLFN11 is oppositely regulated in the ‘responding’ and the ‘non-responding’ cells, indicating that a reduction of SLFN11 indicates a biological response to BGB324.

### Example 8: Screening of Selected Biomarkers in In Vivo Samples from a MOLM13 Systemic Xenograft Model

Material from an in vivo MOLM13 systemic model was also evaluated by flow cytometry. Expression of the previously evaluated biomarkers was examined in cells isolated from bone marrows and spleens of animals with systemic AML disease (MOLM13, inoculated for 7 days prior to treatment) treated with BGB324 at 50 mg/kg for 4 days.

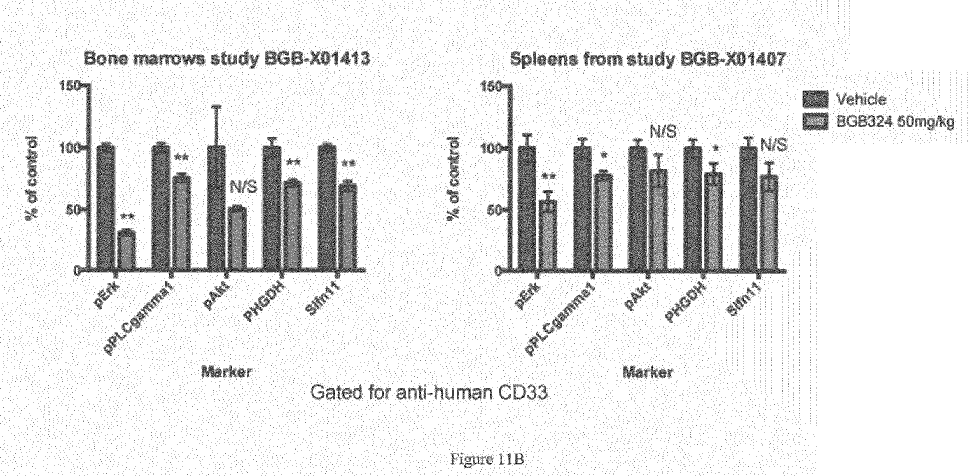

Cells harvested from spleens, blood and bone marrows of the animals were stained with anti CD33 and CD45 antibodies, to determine if systemic disease was established. CD33/CD45-positive cells were identified in spleens (around 10-15%), bone marrows (35-40%) and in the blood (2-6%) of the animals, confirming that the disease was established. There were no significant differences in the percentage of leukemic cells in the BGB324-treated vs. vehicle-treated mice in spleens or bone marrows, but there were a significantly higher percentage of leukemic cells in the blood of BGB324-treated mice (FIG. 11A).

Cells isolated from spleens and bone marrows were assessed for phosphorylation of Erk, PLCγ1 and Akt, and expression of PHGDH and SLFN11. The samples were also co-stained with CD33, and biomarker expression was only evaluated in CD33-positive cells. A significant reduction of pErk, pPLCγ1, PHGDH and SLFN11 in the bone marrows was observed, and significant reduction of pErk, pPLCγ1 and PHGDH in the spleens (FIG. 22B). pAkt when down in both tissues after treatment, but due to a large standard deviation in the control group, this change was not significant.

### INDUSTRIAL APPLICATION

The invention is industrially applicable through operation of methods in accordance with the invention.

1. A method of identifying a subject having an Axl-related condition, the method comprising assessing the level of expression or activity of Slfn11 in the subject, or in a sample derived from the subject.

2. A method according to claim 1 of identifying a subject having a particular risk of developing metastatic or drug-resistant cancer, the method comprising assessing the level of expression or activity of Slfn11 in the subject, or in a sample derived from the subject, an increased level of Slfn11 expression or activity indicating an increased risk of the subject of developing metastatic or drug-resistant cancer.

3. A method according to claim 1 of identifying the presence of a Cancer Stem Cell in a subject, the method comprising determining the level of Slfn11 expression or activity in the subject, or in a sample derived from the subject, increased expression or activity of Slfn11 indicating the existence of a Cancer Stem Cell (CSC).

4. A method according to claim 1 of identifying a subject undergoing epithelial-to-mesenchymal transition (EMT), the method comprising determining the level of Slfn11 expression or activity in the subject, or in a sample derived from the subject, an increase in expression or activity of Slfn11 indicating the occurrence of EMT.

5. A method of prognosing a cancer-related outcome in a subject, the method comprising assessing Slfn11 activity or expression in the subject, or in a sample derived from the subject.

6. A method according to claim 5, wherein:
(i) an increase in Slfn11 activity or expression relative to a control sample is indicative of susceptibility to treatment with an agent capable of inhibiting or reversing EMT, or of increased susceptibility to a chemotherapeutic agent;
(ii) a decrease in Slfn11 activity or expression relative to a control sample is indicative of resistance to treatment with an agent capable of inhibiting or reversing EMT, or of reduced susceptibility to a chemotherapeutic agent;
(iii) a decrease in Slfn11 activity or expression relative to a control sample following treatment of the subject with an Axl or Akt3 inhibitor is indicative of susceptibility to treatment with an agent capable of inhibiting or reversing EMT, or of increased susceptibility to a chemotherapeutic agent; or
(iv) an increase in Slfn11 activity or expression relative to a control sample following treatment of the subject with an Axl or Akt3 inhibitor is indicative of resistance to treatment with an agent capable of inhibiting or reversing EMT, or of reduced susceptibility to a chemotherapeutic agent.

7. A method according to claim 6, wherein the agent capable of inhibiting or reversing EMT is an Axl inhibitor, Akt3 inhibitor, or Slfn11 inhibitor.

8. A method of identifying Axl activity, the method comprising determining the level of Slfn11 expression or activity in the subject, or in a sample derived from the subject, increased expression or activity of Slfn11 correlating with Axl activity.

9. A method according to any one of claims 1 to 8 in which the subject is mammalian.

10. A method according to claim 9 in which the subject is human.

11. A method according to any one of claims 1 to 10, wherein the level of expression or activity in the subject or sample derived from the subject is determined relative to a control sample.

12. A method according to any one of claims 1 to 11, wherein the level of expression of Slfn11 is assessed by determining the copy number of the gene encoding Slfn11 relative to a control sample, wherein an increase in the copy number indicates an increased level of expression of Akt3.

13. A method according to any one of claims 1 to 12, wherein the level of expression of Slfn11 is assessed by determining the level of Slfn11 protein or mRNA.

14. A method of selecting patients, preferably human patients, for treatment of an Axl-related condition, the method comprising identifying patients having elevated Slfn11 activity or expression and selecting thus identified patients for treatment.

15. A method of selecting patients according to claim 14 in which the Axl-related condition is cancer.

16. A method according to any one of claim 14 or 15, wherein the patient is identified according to a method of any one of claims 1 to 13.

17. A method according to any one of claims 14 to 16, wherein the treatment comprises administering an agent capable of inhibiting or reversing EMT.

18. A method according to claim 17, wherein the agent comprises a Slfn11 inhibitor, an Akt3 inhibitor, or an Axl inhibitor.

19. A method according to any one of claims 1 to 23, wherein the cancer or Axl-related condition is a cancer selected from acute myelocytic leukemia (AML), breast, melanoma, prostate, ovarian, colorectal, lung or glioma cancer.

20. An Slfn11 modulator for use in the treatment of an Axl-related condition.

21. An Slfn11 modulator according to claim 20 in which the condition is cancer.

22. An Slfn11 modulator for use in the inhibition of EMT.

23. A compound capable of modulating Slfn11 activity for use in the prevention, inhibition, or treatment of drug resistance in a subject having cancer, the method comprising contacting the subject with a compound capable of modulating Slfn11 activity or expression.

24. A Slfn11 modulator according to any one of claims 21 to 23 in combination with another therapeutic agent.

25. A Slfn11 modulator according to any one of claims 20 to 24, wherein the modulator in an Slfn11 inhibitor.

26. A method of treating a subject having an Axl-related condition, the method comprising contacting the subject with an Slfn11 modulator or pharmaceutical compound selected as, or derived from, a candidate compound obtained by a method according to any one of claims 38 to 42.

27. A method of treatment of a subject according to claim 26 having an Axl-related condition, the method comprising periodically assessing Slfn11 activity or expression in the subject.

28. A method according to one of claims 26 to 27 in which the Axl-related condition is cancer.

29. A method according to one of claims 26 to 28 in which treatment of the subject is adjusted according to detected levels of Slfn11 activity or expression.

30. A method according to any one of claims 26 to 29 in which the subject is being treated with a Slfn11 inhibitor, a Slfn11 activator, an Axl inhibitor, or an Akt3 inhibitor.

31. A method of inhibiting EMT in a subject, the method comprising contacting the subject with a compound capable of inhibiting Slfn11 activity or expression.

32. A method of inhibiting Cancer Stem cells in a subject, the method comprising contacting the subject with a compound capable of inhibiting Slfn11 activity or expression.

33. A method according to any one of claims 26 to 32 in which the subject is also contacted with another cancer therapeutic.

34. A method of preventing or inhibiting drug resistance in a subject having cancer, the method comprising contacting the subject with a compound capable of modulating Slfn11 activity or expression.

35. A method according to any one of claims 26 to 34 in which the subject is mammalian.

36. A method according to claim 35 in which the subject is human.

37. An Slfn11 modulator according to any one of claims 20 to 25, or a method of treatment according to any one of claims 33 to 36 in which the other therapeutic agent is a cancer treatment selected from alkylating agents, including alkyl sulfonates such as busulfan, nitrogen mustards such as chlorambucil, cyclophosphamide, estramustine, ifosfamide, mechlorethamine, melphalan, and uramustine, ethyleneimine derivatives such as thiotepa, nitrosoureas such as carmustine, lomustine, and streptozocin, triazenes such as dacarbazine, procarbazine, and temozolamide, platinum compounds such as cisplatin, carboplatin, oxaliplatin, satraplatin, and picoplatin onnaplatin, tetraplatin, sprioplatin, iproplatin, chloro(diethylenediamino)-platinum (II) chloride, dichloro(ethylenediamino)-platinum (II), diamino(2-ethylmalonato)platinum (II), (1,2-diaminocyclohexane)malonatoplatinum (II), (4-carboxyphthalo)-(1,2-diaminocyclohexane)platinum (II), (1,2-diaminocyclohexane)-(isocitrato)platinum (II), and (1,2-diaminocyclohexane)-cis-(pyruvato)platinum (II); antimetabolites, including antifolates such as methotrexate, permetrexed, raltitrexed, and trimetrexate,pyrimidine analogues such as azacitidine, capecitabine, cytarabine, edatrexate, floxuridine, fluorouracil, gemcitabine, and troxacitabine, and purine analogues such as cladribine, chlorodeoxyadenosine, clofarabine, fludarabine, mercaptopurine, pentostatin, and thioguanine; natural products, including antitumor antibiotics such as bleomycin, dactinomycin, mithramycin, mitomycin, mitoxantrone, porfiromycin, and anthracyclines such as daunorubicin, doxorubicin, epirubicin, idarubicin, and valrubicin, mitotic inhibitors such as the vinca alkaloids vinblastine, vinvesir, vincristine, vindesine, and vinorelbine, enzymes such as L-asparaginase and PEG-L-asparaginase, microtubule polymer stabilizers such as the taxanes paclitaxel and docetaxel, topisomerase I inhibitors such as the camptothecins irinotecan and topotecan, and topoisomerase II inhibitors such as podophyllotoxin, amsacrine, etoposide, teniposide, losoxantrone and actinomycin; hormones and hormone antagonists, including androgens such as fluoxymesterone and testolactone, antiandrogens such as bicalutamide, cyproterone, flutamide, and nilutamide, corticosteroids such as dexamethasone and prednisone, aromatase inhibitors such as aminoglutethimide, anastrozole, exemestane, formestane, and letrozole: estrogens such as diethylstilbestrol, antiestrogens such as fulvestrant, raloxifene, tamoxifen, and toremifine, luteinising hormone-releasing hormone (LHRH) agonists and antagonists such as abarelix, buserelin, goserelin, leuprolide, histrelin, desorelin, nafarelin acetate and triptorelin, progestins such as medroxyprogesterone acetate and megestrol acetate, and thyroid hormones such as levothyroxine and liothyronine; PKB pathway inhibitors, including perifosine, enzastaurin hydrochloride, and triciribine, P13K inhibitors such as semaphore and SF1126, and MTOR inhibitors such as rapamycin and analogues; CDK inhibitors, including seliciclib, alvocidib, and 7-hydroxystaurosporine; COX-2 inhibitors, including celecoxib; HDAC inhibitors, including trichostatin A, suberoylanilide hydroxamic acid, and chlamydocin; DNA methylase inhibitors, including temozolomide, and miscellaneous agents, including altretamine, arsenic trioxide, thalidomide, lenalidomide, gallium nitrate, levamisole, mitotane, hydroxyurea, octreotide, procarbazine, suramin, photodynamic compounds such as methoxsalen and sodium porfimer, and proteasome inhibitors such as bortezomib: molecular targeted therapy agents including: functional therapeutic agents, including gene therapy agents, antisense therapy agents,tyrosine kinase inhibitors such as erlotinib hydrochloride, gefitinib, imatinib mesylate, and semaxanib, Raf inhibitors such as sorafenib, and gene expression modulators such as the retinoids and rexinoids, for example adapalene, bexarotene, trans-retinoic acid, 9-cis-retinoic acid, and N-(4-hydroxyphenyl)retinamide; and phenotype-directed therapy agents, including monoclonal antibodies such as alemtuzumab, bevacizumab, cetuximab, ibritumomab tiuxetan, rituximab, and trastuzumab, immunotoxins such as gemtuzumab ozogamicin, radioimmunoconjugates such as I-tositumobab, and cancer vaccines; Biologic therapy agents including: interferons such as interferon-[alpha]2a and interferon-[alpha]2b, and interleukins such as aldesleukin, denileukin diftitox, and oprelvekin anticancer therapies involving the use of protective or adjunctive agents, including:cytoprotective agents such as amifostine, and dexrazoxane, phosphonates such as pamidronate and zoledronic acid, and stimulating factors such as epoetin, darbeopetin, filgrastim, PEG-filgrastim, and sargramostim; and Axl inhibitor such as 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine; or further combination chemotherapeutic regimens, such as combinations of carboplatin/paclitaxel, capecitabine/docetaxel, fluorauracil/levamisole, fluorauracil/leucovorin, methotrexate/leucovorin, and trastuzumab/paclitaxel, alone or in further combination with carboplatin, and the like.

38. A method of selecting a pharmaceutical compound useful for the prevention, inhibition or treatment of an Axl-related condition, the method comprising providing a group of candidate pharmaceutical compounds for testing, testing the effect of candidate pharmaceutical compounds on Slfn11 activity or expression in a test system, and selecting a candidate pharmaceutical compound on the basis of modulating Slfn11 activity or expression.

39. A method of selecting a candidate pharmaceutical compound useful in the treatment of metastatic or drug resistant cancer, the method comprising providing a group of candidate pharmaceutical compounds for testing, testing the effect of candidate pharmaceutical compounds on Slfn11 activity or expression in a test system, and selecting a candidate pharmaceutical compound on the basis of its modulation of Slfn11 activity or expression.

40. A method of selecting a candidate pharmaceutical compound useful in the prevention or inhibition of EMT, the method comprising providing a group of candidate pharmaceutical compounds for testing, testing the effect of candidate pharmaceutical compounds on Slfn11 activity or expression in a test system, and selecting a candidate pharmaceutical compound on the basis of modulating Slfn11 activity or expression.

41. A method of selecting a candidate pharmaceutical compound useful in the prevention, inhibition or treatment of an Axl-related condition, the method comprising selectively reducing expression of Slfn11 in a test cell, contacting the test cell with the candidate pharmaceutical compound and determining the effect of the candidate pharmaceutical compound on the modulation of Slfn11 activity or expression.

42. A method of selecting a candidate pharmaceutical compound useful in the prevention, inhibition or treatment of an Axl-related condition, the method comprising selectively reducing expression of Slfn11 in an in vitro test system to a low level contacting the test system with a candidate pharmaceutical compound, and selecting candidate pharmaceutical compounds which modulate Slfn11 activity or expression.

43. A method according to any one of claims 38 to 42 in which candidate pharmaceutical compounds which substantially or completely inhibit Slfn11 activity or expression are selected.

44. A method of selecting candidate pharmaceutical compounds according to claim 41, 42 or 43 in which inhibition of Slfn11 activity or expression is indicated by a reduction in EMT.

45. A method according to any one of claims 40 to 44 in which the expression of Slfn11 in cells in the test system is reduced by 90%, 80%, 70%, 60%, 50%, 40%, 30%, or 20%.

46. A method according to claim 45 in which the expression of Slfn11 is reduced so as to not cause inhibition of EMT.

47. A method according to any one of claims 40 to 46 in which the expression of Slfn11 is selectively reduced by introducing into cells in the test system with a nucleotide which interferes with expression of Slfn11.

48. A cell line which is sensitive to inhibitors of EMT, the cell line having a level of Slfn11 expression that is just insufficient to prevent EMT.

49. A cell line according to claim 48 which is a human cell line.

50. A method of identifying a compound which inhibits Slfn11 activity or expression, the method comprising contacting a cell from a cell line according to claim 48 or 49 with a test compound and determining inhibition of Slfn11 activity or expression in the cell.

51. A method according to claim 50 in which inhibition of Slfn11 activity or expression is identified by inhibition of EMT.

52. Use of Slfn11 as a biomarker for detecting the occurrence of epithelial-to-mesenchymal transition (EMT) in a subject.

53. Use according to claim 52 wherein an increase in the expression and/or activation of Slfn11 is indicative of the occurrence of epithelial-to-mesenchymal transition (EMT).

54. Use of Slfn11 as a biomarker for detecting the expression and/or activation of Axl, wherein an increase in the expression and/or activation of Slfn11 is indicative of an increase in the expression and/or activation of Axl.

55. A method for detecting the occurrence of epithelial-to-mesenchymal transition (EMT) in a sample, said method comprising
determining the expression level or activation of Slfn11 in a sample isolated from a cell, group of cells, an animal model or human as compared to a control sample, wherein an increase in the expression level or activation of Slfn11 relative to the control sample is indicative of the occurrence of epithelial-to-mesenchymal transition (EMT).

56. A method for identifying an agent capable of inhibiting or reversing epithelial-to-mesenchymal transition (EMT), said method comprising administering said agent to a cell, group of cells or animal model, and monitoring the activation and/or the expression of Slfn11.

57. A method according to claim 56 which comprises:
(i) administering the agent to a cell, group of cells or an animal model, not a human; and
(ii) measuring Slfn11 expression and/or Slfn11 activation in samples derived from the treated and the untreated cells or animal model; and
(iii) detecting an increase in the expression and/or activation of Slfn11 in the treated sample as compared to the untreated sample as an indication of the ability to inhibit or reverse epithelial-to-mesenchymal transition (EMT).

58. A method according to claim 56 or claim 57, wherein the animal model is not a human.

59. A use or method according to any one of claims 53 to 58 wherein the level of expression of Slfn11 is assessed by determining the copy number of the gene encoding Slfn11 relative to a control sample, wherein an increase in the copy number indicates an increased level of expression of Slfn11.

60. A use or method according to any one of claims 52 to 59 wherein the level of expression of Slfn11 is assessed by determining the level of Slfn11 protein or mRNA.
